(19) United States
(12) Patent Application Publication
(10) Pub. No.: US 2005/0059055 A1
(43) Pub. Date: Mar. 17, 2005

(54) Fast accurate evaluation of solvent exposure

(75) Inventors: Naigong Zhang, Arlington, VA (US); Chen Zeng, Rockville, MD (US); Ned S. Wingreen, Princeton, NJ (US)

Correspondence Address: DARBY & DARBY P.C., P. O. BOX 5257, NEW YORK, NY, 10150-5257, US

(73) Assignee: NEC Laboratories America, Inc., Princeton, NJ (US)

(21) Appl. No.: 10/901,008

(22) Filed: Jul. 26, 2004

Related U.S. Application Data
(60) Provisional application No. 60/501,547, filed on Sep. 9, 2003.

(51) Int. Cl. C12Q 1/68

(52) U.S. Cl. 435/6; 702/20

(57) ABSTRACT

The present invention relates to a method for quantitative design and optimization of polymers. More specifically, the present invention relates to method for calculating the solvent-exposed area of a polymer.

This application claims priority under 35 U.S.C. §119(e) to co-pending U.S. Provisional Patent Application Ser. No. 60/501,547 (filed Sep. 9, 2003).

Numerous references, including patents, patent applications and various publications are cited and discussed in this specification. The citation and/or discussion of such references is provided to clarify the description of the invention and is not an admission that any such reference is “prior art” to the invention described herein. All references cited and discussed in this specification are incorporated by reference in their entirety and to the same extent as if each reference was individually incorporated by reference.

### 1. FIELD OF THE INVENTION

The present invention relates to a method for quantitative design and optimization of polymers. Furthermore, the present invention relates to biomolecular engineering, the design of biopolymers such as proteins and nucleic acids, wherein an “inverse protein folding” approach is adopted and to folding simulations of biopolymers such as proteins and nucleic acids. In particular, the invention relates to computational methods for estimating the solvent-exposed surface area of polymers.

### 2. BACKGROUND OF THE INVENTION

This invention is concerned with polymers, primarily biopolymers such as polynucleotides (chains of nucleic acids, e.g. DNA and RNA) and polypeptides (chains of amino acids, e.g. proteins). The methods of the present invention are directed to quantitative design and optimization of biopolymers, preferably proteins, using an “inverse protein folding” approach. The present invention improves on current methods utilized for protein design and optimization. In particular, the present invention provides a fast and accurate method for estimating the solvent-exposed area of polymers on a residue by residue basis.

The inverse folding problem was originally defined by Drexler (Drexler, K. E., Proc. Natl. Acad. Sci. (USA) 1981, 78:5275-5278 and Pabo (Pabo, Nature 1983, 301, 200) as the problem of defining the sequences compatible with a given protein fold. It is fundamental to protein design and engineering, and, as such, has attracted considerable interest. Mutter, et al., Cell. Mol. Life Sciences 1997, 53:851-863; Smith, et al., Accts. Of Chem. I Res. 1997, 30:153-161;. Cao, et al., Prog, In Biochem. and Biophys. 1998, 25:197-201; Giver, et al., Cur. Opin. Chem. Biol. 1998, 2:335-338; Regan, et al., Curr. Opin. Struct. Biol. 1998, 8:441-442; Shakhnovich, E. I., Folding & Design 1998, 3:R45-R58. Since the function of a protein is directly related to its three dimensional structure, manipulation of the structure via sequence changes can provide functional diversity. Protein molecules can be engineered to optimize their activities, as well as to alter their pharmacokinetic properties.

The three-dimensional structure and activity of a protein is a strong function of solvent. The importance of a solvent, particularly water, for the structure and activity of proteins has long been recognized (Franks, F; Biophys. Chem. 2002 96:117-127). Indeed, proteins lack activity in the absence of water. In solution proteins possess a conformational flexibility and can adopt a large number of conformational states. Equilibrium between these conformational states will depend on the interaction of the solvent with the protein (Parsegian, V. A; Int. Rev. Cytology 2002, 215:1-31). Moreover, the burial of hydrophobic residues away from the aqueous solvent is a major determinant of specific folding (Dill, K. A.; Biochemistry 1990; 356:539-542). Consequently, it is no surprise that accurate solvation energies are needed for both accurate folding simulations and for design of a protein having desired properties.

Historically, the major obstacle to developing and implementing fast and accurate protein design algorithms is the large number of possible sequences for a polypeptide with m possible residues. A polypeptide with backbone of length n with m possible sidechains per position will have mⁿ possible sidechain sequences, a number which grows exponentially with sequence length. The large number of possible protein sequences can render calculations for protein design either unwieldy or impossible in real time. Recently, a number of fast search algorithms based on combinatorial optimization have been utilized to solve this combinatorial search problem (Hellinga, et al., J. Mol. Biol. 1991, 222:763-785; Hurley, et al., J. Mol. Biol. 1992, 224:1143-1154; Desjarlaisl, et al., Protein Science 1995, 4:2006-2018; Harbury, et al., Proc. Natl. Acad. Sci. USA 1995, 92:8408-8412; Klemba, et al., Nat. Struc. Biol. 1995, 2:368-373; Nautiyal, et al., Biochemistry 1995, 34:11645-11651; Betzo, et al., Biochemistry 1996, 35:6955-6962; Dahiyat, et al., Protein Science 1996, 5:895-903; Jones, Protein Science 1994, 3:567-574; Konoi, et al., Proteins: Structure, Function and Genetics 1994, 19:244-255).

One important application is de novo protein design. In a common de novo design strategy amino acid residues may be modeled as sidechains that interact with a fixed backbone. For fixed-backbone de novo protein design, the “dead-end-elimination” (DEE) algorithm has been proven highly efficient (Desmet, et al., Nature 1992, 356:539-542). “Dead-end elimination” is a deterministic search algorithm that seeks to systematically eliminate bad sidechains (where “sidechain” includes the chemical species and spatial conformation) and combinations of sidechains until a single solution remains. The DEE calculation is based on the fact that if the worst total interaction of a first sidechain is still better than the best total interaction of a second sidechain, then the second sidechain cannot be part of the global optimum solution. Since the energies of all sidechains have already been estimated, the DEE approach only requires sums over the sequence length to test and eliminate sidechains, which speeds up the calculations considerably. A requirement for successful application of the DEE algorithm is, the existence of an accurate pairwise approximation for the total protein exposure and burial of polar and nonpolar atoms, separately (see Desmet, et al., supra).

For further discussion of this method see, Goldstein, R. F., Biophys. J. 1994, 66:1335-1340; Desmet et al., Nature 1992, 356:539-542; De Maeyer, et al., Fold. Des. 1998, 2:53-56. Gordan, et al., J. Comp. Chem. 2000, 19:1505-1514; Pierce, et al., J. Comp. Chem. 2000, 21:999-1009.

In practice, accurate solvation energies depend on accurate calculation of solvent exposure. Solvation-free energies of nonpolar molecules are observed to be roughly proportional to the solvent-accessible surface area of the molecules (Hermann, R. B., J. Phys. Chem. 1972, 76:2754. However, exact evaluation of exposure is numerically demanding, and may become a computational bottleneck. From the point of view of the computational efficiency, it would be very attractive to approximate the solvent-exposed surface area of a protein by the sum of pairwise interaction of atoms in the protein. The difficulty in obtaining an accurate pairwise approximation to surface areas lies in treating multiple-atom, multiple-residue buried areas. For two spheres in contact, there is a simple analytical formula to calculate the buried area (and therefore the exposed area) using the two radii and the separation between the spheres (Wodak, et al., J. Proc. Natl. Acad. Sci. 1980, 77:1736-1740). For proteins, this formula was initially applied to each pair of atoms, and the total exposed and buried areas were estimated via a statistical combination (see Wodak et al., supra).

Street and Mayo significantly improved on prior known methods for the statistical combination of pair-atom areas by calculating pair-residue areas (Street, et al., Fold. Des. 1998; 3:253-258). In the Street and Mayo method, two residues were placed at positions i and j, first separately and then together, and the areas buried by the two sidechains were estimated by taking into account the presence of the backbone. Because buried areas often overlap, a straightforward addition of pair-residue areas overestimates the total buried area. Consequently, Street and Mayo scaled down the pairwise buried area by an adjustable factor. They went further and used three pairwise scaling factors for the residues classified into core and non-core: 0.42 for the interaction of core/core residues, 0.79 for non-core/non-core residues, and 0.74 for core/non-core residues. Street and Mayo classified residues as core or non-core using an algorithm that considered the direction of each sidechains's C_{α}-C_{β} vector relative to a surface computed using only the template C_{α} atoms with a carbon radius of 1.95 Å, a probe radius of 8 Å and no add-on radius. A residue was classified as a core position if both the distance from its C_{α} atom (along its C_{α}-C_{β} vector) to the surface was greater than 5.0 Å and the distance from its C_{β} atom to the nearest point in the surface was greater than 2.0 Å (Marshall, et al., J. Mol. Biol. 2001, 305:619-631). The scaling factor for the two core residues was smaller than the scaling factors with non-core residues involved because the overlapping burial effect is stronger in the core (see Street et al, supra). Despite the use of specialized scaling factors, the accuracy of the Street and Mayo method was still limited by the effect of overlapping burial of core residues.

The present invention improves upon known methods for designing and optimization proteins using an inverse folding approach. In particular, the present invention calculates the solvent-exposed area of polymers, preferably proteins, by incorporating the many-residue burial effect directly in the estimations of the single-residue and pair-residue areas. Instead of calculating these areas by taking into account the presence of the backbone alone, the present invention calculates the areas by taking into account the presence of the backbone and generic sidechains. Each of these generic sidechains may consist of one or a number of spheres or other geometrical shapes and may be optimized to approximate closely the presence of real sidechains. The generic sidechains may be optimized for a subset of polymers whose buried or exposed areas are known, or for a subset of polymers whose buried or exposed areas can be easily calculated utilizing exact methods described below. The optimized generic sidechains may then be utilized to predict the buried or exposed areas of polymers whose exposed or buried areas are unknown. The essential advantage of the present invention is that much of the overlapping burial effect is automatically accounted for by the genericized sidechains. As a result, errors in the area calculations are dramatically reduced, and there is no need to optimize scaling factors—they can be set to one.

### 3. SUMMARY OF THE INVENTION

In accordance with the objects outlined above, the present invention improves on current methods that utilize an inverse folding approach to polymer design and optimization. In particular, the present invention provides a fast and accurate pairwise method for estimating the solvent-exposed surface area of polymers, preferably, biopolymers, such as proteins and nucleic acids. The methods are straightforward and are computationally tractable. Applicants have discovered that estimating the single-residue and pair-residue surface areas by taking into account the presence of both the backbone and generic sidechains decreases the errors associated with solvent-exposed area calculations without a large computational cost.

The invention therefore provides methods for estimating the single-residue and pair-residue surface areas by taking into account the presence of both the backbone and generic sidechains and determining the exposed and buried areas by a combination of these single-residue and pair-residue areas. The methods further comprise estimating the single-atom and pair-atom surface areas by taking into account the presence of both the backbone and generic sidechains. Generally, the generic sidechains may be chosen from simple geometric shapes, such as a spheres, so as to minimize the difference in the exact surface area and the estimated surface area. However, the use of other geometric shapes is not foreclosed.

Computer systems are also provided that may be used to estimate the exposed-surface area of polymers. These computer systems comprise a processor interconnected with a memory that contains one or more software components. In particular, the one or more software components include programs that cause the processor to implement the steps of the analytical methods described herein. The software components may comprise additional programs and/or files including, for example, sequence or structural databases of polymers, preferably biopolymers, such as proteins.

Computer program products are further provided, which comprise a computer readable medium, such as one or more floppy disks, compact discs (e.g., CD-ROMS or RW-CDS), DVDs, data tapes, etc) that have one ore more software components encoded thereon in computer readable form. In particular, the software components may be loaded into the memory of a computer system and then cause a processor of the computer system to execute steps of the analytical methods described herein. The software components may include additional programs and/or files including databases, e.g., of polymer sequences and/or structures.

### 4. BRIEF DESCRIPTION OF THE DRAWINGS

FIG. 1 is a flow diagram illustrating an exemplary embodiment of the methods of the invention.

FIG. 2 is a schematic representation of a protein comprising amino acid residues. The backbone atoms 10 (indicated by solid circles), the backbone unit 20 (the dashed rectangle), the real sidechains 30, and the generic sidechains 40. Each backbone unit is covalently bonded to its immediately adjacent backbone units.

FIG. 3 is a schematic representation of the Street and Mayo method for pairwise calculation of the exposed exposed surface area (Eqn. 2). The real sidechains are depicted as a rectangle 30 against the backbone 40. In FIG. 3A

$${A}_{i , bb}^{exposed}$$

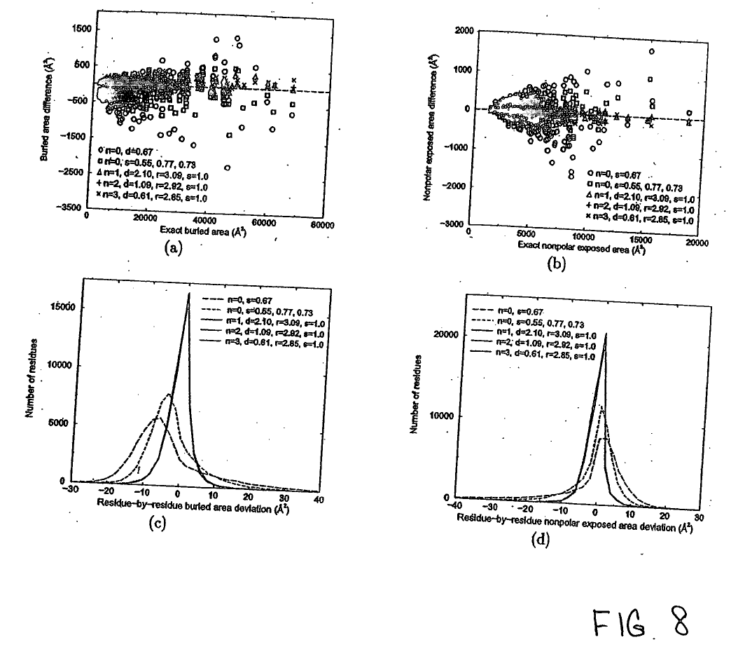

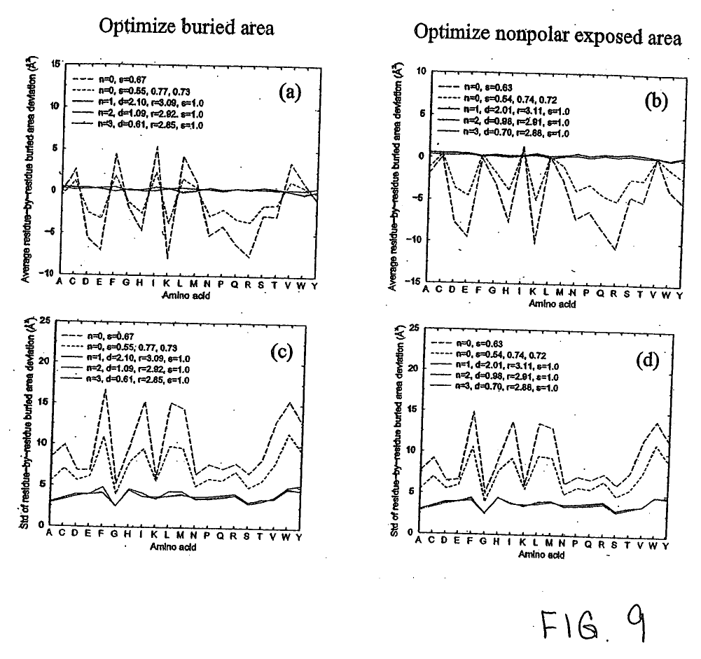

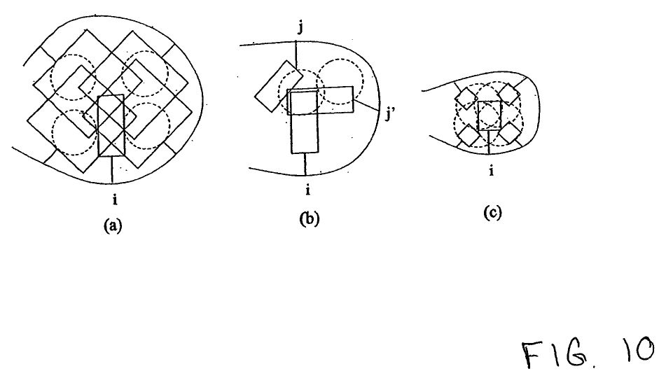

(indicated by bold line with arrows), the exposed area of the sidechain at location i (along the backbone) is depicted in the presence of the backbone; In FIG. 3B

$${A}_{j , bb}^{exposed}$$

(indicated by bold line with arrows), the exposed area of the sidechain at location j is depicted in the presence of the backbone; In FIG. 3C

$${A}_{ij , bb}^{exposed}$$

(indicated by bold line with arrows), the total exposed area of sidechains at i and j each is depicted in the presence of the backbone. The dashed line with arrows shows

$${A}_{i , bb}^{exposed} + {A}_{j , bb}^{exposed} - {A}_{ij , bb}^{exposed}$$

which is the area buried by the sidechains at i and j. This buried area is subtracted from

$${A}_{i , bb}^{exposed} + {A}_{j , bb}^{exposed}$$

in Eq. 2, with a scaling factor s; In FIG. 3D multiply-buried area (dotted line), which would be overcounted if s=1.0 was used in Eq. 2 is depicted.

FIG. 4 is a schematic representation of the present invention's generic-sidechain method for pairwise estimation of exposed surface areas. The real sidechains 30 are depicted as rectangles (which represent an arbitrary shape in three dimensions) and generic sidechains 50 are depicted as circles (which respresents spheres in three dimensions). Both real sidechains 30 and generic sidechains 50 are shown against the backbone 40. In FIG. 4A

$${A}_{i , gs}^{exposed}$$

(indicated by the bold line with arrows), the exposed area of the sidechain at i is depicted in the presence of the backbone and generic sidechains at other positions; In FIGS. 4B and 4C

$${A}_{i : j , gs}^{exposed}$$

(indicated by the solid line with arrows), the exposed area of the sidechain at i is depicted in the presence of the real sidechain at j, the backbone, and generic sidechains at all other positions. In FIG. 4B the real sidechain at j is large and covers more area than the generic sidechain at j, hence

$${A}_{i : j , gs}^{exposed} - {A}_{i , gs}^{exposed}$$

is negative. In FIG. 4C the real sidechain at j is small and covers less area than the generic sidechain, hence

$${A}_{i : j , gs}^{exposed} - {A}_{i , gs}^{exposed}$$

is positive. In Eq. 3,

$${A}_{i , gs}^{exposed}$$

is corrected by

$$s {\sum }_{i , j \neq i}^{} ( {A}_{i : j , gs}^{exposed} - {A}_{i , gs}^{exposed} ) .$$

We find that s=1.0 is optimal, i.e., there is no need for a scaling factor s because the overlapping burial effect is automatically adequately accounted for by the generic sidechains. (However, the use of a scaling factor is not foreclosed.)

FIG. 5 shows as exemplary computer system that may be used to implement analytical methods of this invention.

FIG. 6 is a schematic representation of three different emboidments of the present invention's generic sidechain method as shown in FIG. 4, wherein the backbone is horizontal. The three alternative implementations of generic sidechains (one sphere, two spheres, and three spheres) shown in FIG. 5 are not exhaustive of all the possibilities. The generic sidechains 50 are depicted as spheres drawn to scale with optimized parameters d and r. d is the distance from the C_{α} atom, which comprises the backbone, to the center of the first sphere and the separation between adjacent spheres; r is the radius of the generic-sidechain spheres against the backbone 40. (a) one sphere (n=1) with d=2.10 Å and r=3.09 Å; (b) two spheres (n=2) with d=1.09 Å and r=2.92 Å; (c) three spheres (n=3) with d=0.61 Å and r=2.85 Å. The thick black line represents the protein backbone 40.

FIG. 7A-D show surface-area results for the 10-protein set of Street and Mayo: for each protein in the set, FIG. 7A is a plot of the difference between the total pairwise buried area,

$${A}_{pairwise}^{buried} ,$$

and the exact buried area,

$${A}_{exact}^{buried} ,$$

plotted against the exact buried area, FIG. 7B is a plot of the difference between the total pairwise nonpolar exposed area,

$${A}_{pairwise}^{( n ) exposed} ,$$

and the exact nonpolar exposed area,

$${A}_{exact}^{( n ) exposed} ,$$

against the exact nonpolar exposed area. FIG. 7C shows distributions of residue-by-residue buried-area deviations

$$δ {A}_{i , pairwise}^{buried} = {A}_{i , pairwise}^{buried} - {A}_{i , exact}^{buried}$$

(Eq. 10) for all residues in the 10-protein set. FIG. 7D shows distributions of residue-by-residue nonpolar-exposed-area deviations

$$δ {A}_{residue}^{( n ) exposed} ( i ) = {A}_{i , pairwise}^{( n ) exposed} - {A}_{i , exact}^{( n ) exposed}$$

for all residues.

FIGS. 8A-D show surface-area results for the 377 CATH proteins: for each protein, FIG. 8A is a plot of the difference between the total pairwise buried area,

$${A}_{pairwise}^{buried}$$

and the exact buried area,

$${A}_{exact}^{buried} ,$$

against the exact buried exact area; FIG. 8B is a plot of the difference between the total pairwise nonpolar exposed area,

$${A}_{pairwaise}^{( n ) exposed} ,$$

and the exact nonpolar exposed area,

$${A}_{exact}^{( n ) exposed} ,$$

against the exact nonpolar exposed area. FIG. 8C, shows the distributions of residue-by-residue buried-area deviations

$$δ {A}_{residue}^{buried} ( i ) = {A}_{i , pairwise}^{buried} - {A}_{i , exact}^{buried}$$

for all residues in the 377 CATH proteins; and FIG. 8D shows distributions of residue-by-residue nonpolar-exposed-area deviations for all

$$δ {A}_{residue}^{( n ) exposed} ( i ) = {A}_{i , pairwise}^{( n ) exposed} - {A}_{i , exact}^{( n ) exposed}$$

residues.

FIGS. 9A-D show deviations as follows: FIGS. 9A and 9B show the average deviation and FIGS. 9C and 9D show the standard deviation of the residue-by-residue buried area deviation for each amino-acid type, averaged over the residues of the 377 CATH proteins. The parameters have been optimized for the 10-protein set of Street and Mayo using total buried area in FIGS. 9A and 9C and using total nonpolar exposed area in FIGS. 9B and 9D.

FIG. 10 is a schematic illustration of the three sources of important or potentially important error in the generic-sidechain approximation. In FIG. 10A, part of the residue at location i on the backbone is multiply-buried by real sidechains (rectangles) at other sites, but not by the generic sidechains (circles). This results in a large overestimate for the buried area of residue i. In FIG. 10B part of the residue at i is buried by one (and only one) generic sidechain but not by the real sidechain (at j), and the same part of i is also buried by another real sidechain (at j′). This results in an underestimate of the buried area at i. In FIG. 10C, the residue at i is multiply-buried by the generic sidechains at other sites, but not by real sidechains. This also results in an overestimate for the buried area at i.

### 5. DETAILED DESCRIPTION OF THE INVENTION

The invention overcomes problems in the prior art and provides novel methods which can be used for quantitative design and optimization of polymers, preferably proteins, using an inverse protein folding approach, which seeks the optimal sequence for a desired structure. Inverse folding is similar to protein design, which seeks to find a sequence or a set of sequences that will fold into a desired structure. The invention may also be used for protein threading technique, the problem of aligning a protein sequence to a given structural model. These approaches can be contrasted with a protein folding approach which attempts to predict a structure adopted by a given sequence. In particular, the invention provides a faster and more accurate pairwise method for determining the solvent-exposed surface area of polymers. For example, by incorporating the many-residue burial effect directly in the single-residue and pair-residue areas, errors in the area calculations are dramatically reduced without a corresponding increase in computational cost. Other advantages of the invention will be apparent below.

Details of the invention are described below, including specific examples. These examples are provided to illustrate embodiments of the invention. However, the invention is not limited to the particular embodiments, and many modifications and variations of the invention will be apparent to those skilled in the art. Such modifications and variations are also part of the invention.

5.1 Overview of Modeling Techniques

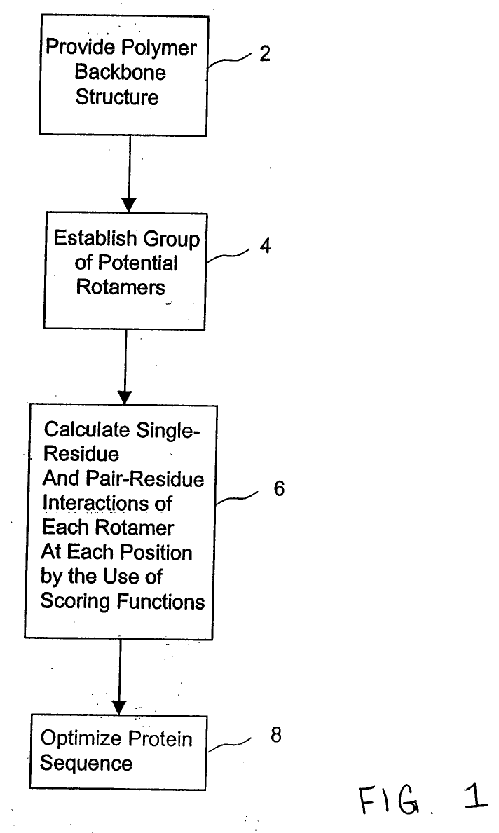

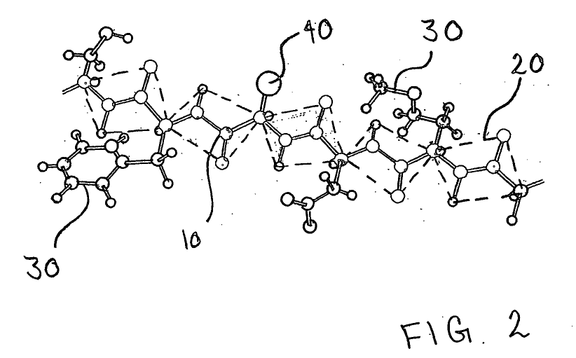

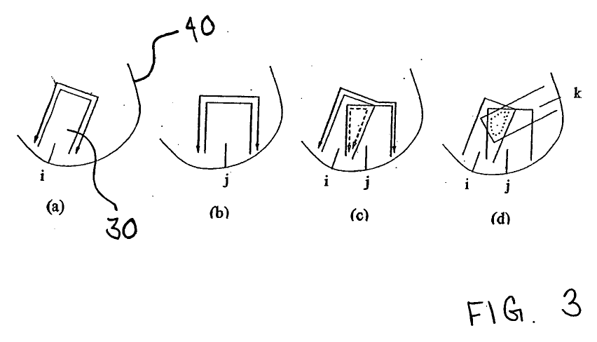

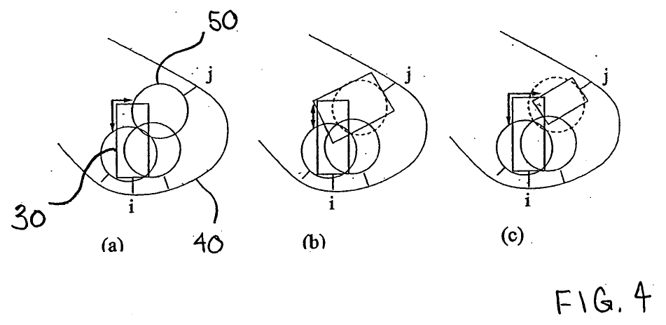

According to the invention, computational methods are utilized to design and optimize polymers, preferably biopolymers, such as proteins and nucleic acids. FIG. 1 is a flow diagram illustrating exemplary embodiment of the invention. The general preferred approach of the present invention is as follows, although alternate embodiments are discussed below. A known polymer backbone structure is used as the starting point. This is shown as step 2 in FIG. 1. The residues to optimized are then identified, which may be the entire sequence or subset(s) thereof. The side chains of any position to be varied are then removed. Each residue can be represented by a discrete set of all allowed conformers of each side chains, called rotamers. This is shown as step 4 of FIG. 1. Thus, to arrive at an optimal sequence for a backbone, all possible sequences of rotamers must be screened, where each backbone position can be occupied either by each sidechain in all its possible rotameric states, or a subset of sidechains, and thus a subset of rotamers.

In a preferred embodiment, two interactions are then calculated for each rotamer at every position: the “single-residue” interaction (i.e., a single residue pretending there are no real sidechains for any of the other residues to be varied) and the “pair-residue” interaction (i.e., a pair of residues taking into account the effect of the sidechain of the other residue in the pair). The energy of each of these interactions is calculated through the use of a variety of scoring functions, which include the energy of van der Waal's forces, the energy of hydrogen bonding, the energy of secondary structure propensity, the energy of surface area solvation and the electrostatics. This is shown as step 6 in FIG. 1. Thus, the total energy of each rotamer interaction is calculated, and stored in a matrix.

In a preferred embodiment, the energy of surface area solvation is directly proportional to the solvent-exposed surface area of the nonpolar atoms of the polymer. In a preferred embodiment, the solvent-exposed surface area is determined by the calculation of two components, single-residue area and pair-residue surface areas. In a preferred embodiment, the single-residue and pair-residue surface areas are estimated taking into account the presence of both the backbone and generic sidechains as is more fully described below.

The discrete nature of rotamer sets for which rotamer interactions are calculated allows a simple calculation of the number of rotamer sequences to be tested. A backbone of length n with m possible rotamers per position will have mⁿ possible rotamer sequences. The large number of possible protein sequences can render calculations for polymer design either unmanageable or impossible in real time. A number of well-known fast search algorithms based on combinatorial optimization can be utilized to solve this combinatorial search problem (Hellinga, et al., J. Mol. Biol. 1991, 222:763-785; Hurley, et al., J. Mol. Biol. 1992, 224:1143-1154; Desjarlaisl, et al., Protein Science 1995, 4:2006-2018); Harbury, et al., Proc. Natl. Acad. Sci. USA 1995, 92:8408-8412; Klemba, et al., Nat. Struc. Biol. 1995, 2:368-373; Nautiyal, et al., Biochemistry 1995, 34:11645-11651; Betzo, et al., Biochemistry 1996, 35:6955-6962; Dahiyat, et al., Protein Science 1996, 5:895-903; Jones, Protein Science 1994, 3:567-574; Konoi, et al., Proteins: Structure, Function and Genetics 1994, 19:244-255). This is shown as step 8 in FIG. 1.

Once the global solution has been found, the results may then be experimentally verified by physically generating one or more of the polymer sequences followed by experimental testing. The information obtained from the testing can then be fed back into the analysis, to modify the procedure if necessary.

Thus, the present invention provides a method of designing or optimizing polymers, preferably proteins. The method comprises providing a polymer backbone structure with variable residues positions, and then establishing a group of potential rotamers for each of the residue positions. The interactions between the polymer backbone and the potential rotamers, and between pairs of potential rotamers, are then processed to generate a set of optimized polymer sequences, preferably a single global optimum, which then may be used to generate other related sequences.

In other embodiments, the computational methods of the present invention are applied to design a multiple polymers (e.g. 2, 3, 4 or more) or polymer assembly. In one embodiment, a polymer-polymer complex is designed and optimized. Suitable polymer-polymer complex includes, but are not limited to, a ligand-protein, protein-lipid, protein-protein, protein-nucleic acid, and protein-enzyme complexes. Alternatively, the computational methods of the present invention are applied to design and optimize a single polymer comprising a polymer-polymer complex. For example, the sidechains of a ligand are optimized wherein the ligand is bound to a protein.

Polymer Backbone

The term “polymer” means any substance or compound that is composed of two or more building blocks (‘mers’) that are repetitively linked together. For example, a “dimer” is a compound in which two building blocks have been joined together; a “trimer” is a compound in which three building blocks have been joined together; etc. The individual building blocks of a polymer are also referred to herein as “residues”.

Suitable polymers include branched polymers, such as poly-4-methyl pentene-1, branched polypropylene, and branched polystyrene.

Preferably the polymer is a biopolymer, preferably a protein. By “biopolymer” herein is meant any polymer having an organic or biochemical utility or that is produced by a cell. Preferred biopolymers include, but are not limited to, polynucleotides, polypeptides or proteins and polysaccharides.

By “protein” herein is meant at least three amino acids linked together by a peptide bond. Thus, as used herein, “protein” includes proteins, oligopeptides and peptides. The peptidyl group may comprise naturally occurring amino acids and peptide bonds, or synthetic peptidominetic structures, i.e. “analogs”, such as peptoids (see Simon, et al., Proc. Natl. Acad. Sci. 1992, 89(20):9367). The amino acids may either be naturally occurring or non-naturally occurring; as will be appreciated by those in the art, any structure for which a set of rotamers, (i.e., sidechains of specific conformation) is known or can be generated can be used in the same manner as an amino acid in the method of the present invention.

The proteins may be from any organism, including prokaryotes and eukaryotes, including, without limitation, enzymes from bacteria, fungi, extremeophiles such as the archebacteria, insects, fish, animals (particularly mammals and particularly humans) and birds.

Suitable proteins include, but are not limited to, industrial and pharmaceutical proteins, including ligands, cell surface receptors, antigens, antibodies, cytokines, hormones, and enzymes. Suitable classes of enzymes include, but are not limited to, hydrolases such as proteases, carbohydrases, lipases,; isomerases such as racemases, epimerases, tautomerases, or mutases; transferases, kinases, oxidoreductases, and phophatases. Suitable enzymes are listed in the Swiss-Prot enzyme database.

Specifically included within “protein” are fragments and domains of known proteins, including functional domains such as enzymatic domains, binding domains, etc., and smaller fragments, such as turns, loops, etc. that is, protein segments of proteins may be used as well. The segments of proteins must comprise of at least three amino acid residues.

Once the polymer is chosen, the polymer backbone structure is input into the computer. This operation is shown as step 2 in FIG. 1. By “polymer backbone structure” or grammatical equivalents herein is meant the three dimensional coordinates that defined the three dimensional structure of a particular polymer. Suitable polymer backbones include, but are not limited to, all of those found in the protein database compiled and serviced by the Brookhaven National Lab. Examples of protein backbones found in the protein database compiled and serviced by the Brookhaven National Lab include, but are not limited to, the protein backbones of lysozyme, albumin, hemoglobin, and myoglobin. For a protein, the components which comprise the polymer backbone structure (of a naturally occurring protein) are nitrogen, the carbonyl carbon, the α-carbon (C_{α}), and the carbonyl oxygen, along with the direction of the vector from the α-carbon to the β-carbon (C_{β}). For proteins, all other moieties of each amino acid residue are sidechains.

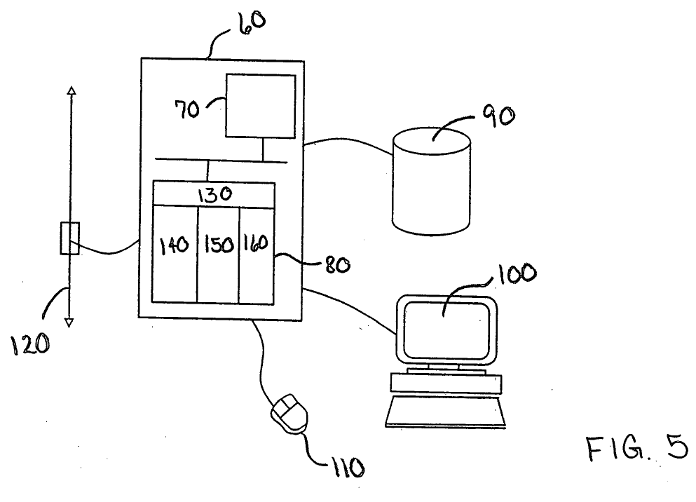

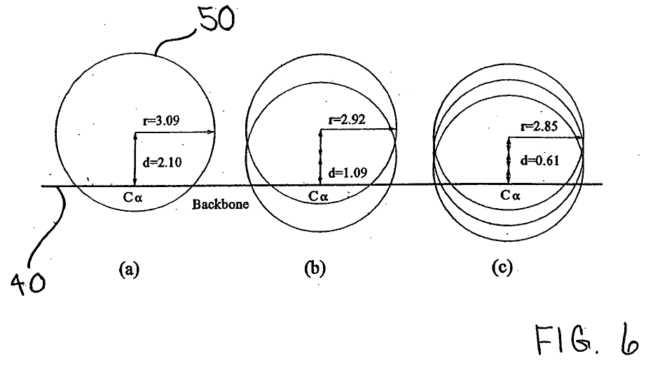

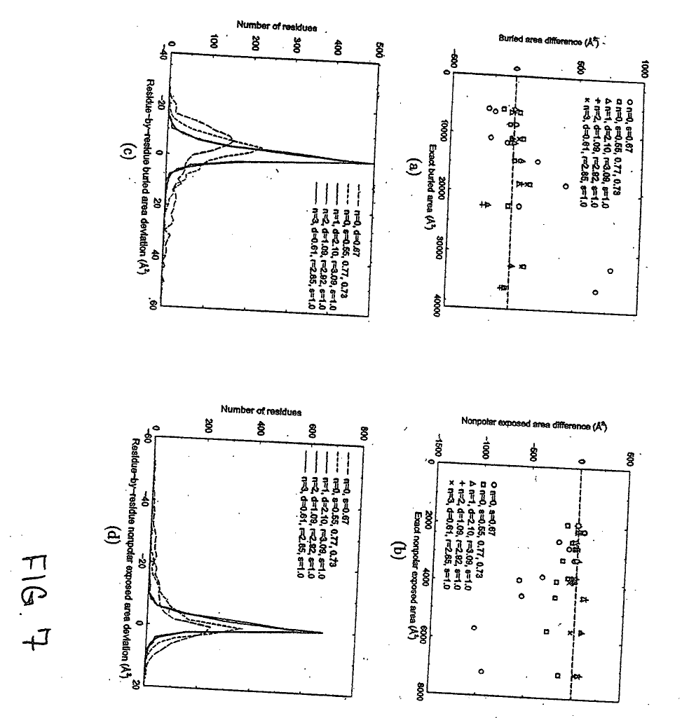

For a protein, an individual building block or residue is the part of the backbone and the sidechain derived from a single amino-acid molecule (see FIG. 2). The structures which comprise a backbone structure 20 (of a naturally occurring protein) are nitrogen, the carbonyl carbon, the α-carbon, and the carbonyl oxygen, along with the direction of the vector from the α-carbon to the β-carbon. The atoms that comprise the sidechains 30 structure (of a naturally occurring protein) include, but are not limited to carbon, oxygen, nitrogen, sulfur, and selenium. The sidechain structure may also be “generic”. Generic sidechains are simplified representations of real sidechains. By “generic” is meant a non-naturally occurring sidechain, preferably chosen from the general class of “simple geometric shapes”. By “simple geometric shapes” is meant the three-dimensional structures resulting from the revolution of “non-complex geometric shapes” around an axis. “Non-complex geometric shapes” include, but are not limited to a circle, a square, a triangle, a rectangle and an ellipse. Generic sidechains 40 (actually 2-dimensional projections thereof) are also as shown in FIG. 2.

The chosen polymer may be any polymer for which a three dimensional structure is known or can be generated; that is, any polymer for which there are three dimensional coordinates for the atoms of the polymer. The time of elucidation of the 3-D structure of the polymer is not relevant to the scope of the present invention. As further 3-D structures are elucidated, the method can be applied to these structures. Generally, 3-D structures can be determined using X-ray crystallographic techniques, NMR techniques, de novo modeling, homology modeling, etc. If X-ray structures are used, structures at 2 Å resolution or better are preferred, but not required. Moreover, if de novo modeling is used, the polymer backbone could be designed from scratch, possibly independent of any sequence.

The polymer backbone structure contains at least one variable residue position. By “variable residue position” herein is meant a residue position of the polymer to be designed wherein the sidechain or rotamer of the residue is not fixed in the design method as a specific sidechain or rotamer.

In a preferred embodiment, all the residue positions of the polymer are variable. That is, every sidechain may be altered in the methods of the present invention. In an alternate preferred embodiment, only some of the residue positions of the polymer are variable, and the remainder are “fixed”, that is, they are identified in the three dimensional structure as being in a set conformation. In some embodiments, a fixed position is left in its original conformation (which may or may not correlate to a specific rotamer of the rotamer library being used). In a preferred embodiment, residues which can be fixed include, but are not limited to, structurally or biologically functional residues. For example, residues which are known to be important for biological activity of a protein, such as the residues which form the active site of an enzyme, the substrate binding site of an enzyme, the binding site for a binding partner (ligand/receptor, antigen/antibody, etc.), phosphorylation or glycosylation sites which are crucial to biological function, or structurally important residues, such as disulfide bridges, metal binding sites, critical hydrogen bonding residues, residues critical for backbone conformation such as proline or glycine, residues critical for packing interactions, etc. may all be fixed in a conformation or as a single rotamer.

Similarly, residues which may be chosen as variable residues may be those that confer undesirable biological attributes, such as susceptibility to proteolytic degradation, dimerization or aggregation sites, glycosylation sites which may lead to immune responses, unwanted binding activity, unwanted allostery, undesirable enzyme activity but with a preservation of binding, etc.

Once the protein backbone structure has been selected and input, and the variable residue positions chosen, a group of potential rotamers for each of the variable residue positions is established. This is shown as step 4 in FIG. 1. In one embodiment of the invention, a group of potential rotamers for each of the variable residue positions in chosen from a rotamer library, as described below.

Rotamer Library

As is known in the art, each amino acid side chain has a set of possible conformers, called rotamers. See Ponder, et al., Acad. Press Inc. (London) Ltd. 1987, 775-791; Dunbrack, et al., Struc. Biol. 1994, 195:334-340; Desmet, et al., Nature 1992, 356:539-542. Thus, a set of discrete rotamers for every amino acid side chain is used. There are two general types of rotamer libraries: backbone dependent and backbone independent. A backbone dependent rotamer library allows different rotamers depending on the position of the residue in the backbone. For example, for a protein, certain leucine rotamers are allowed if the position is within an α-helix, and different leucine rotamers are allowed if the position is not in a α-helix. A backbone independent rotamer library utilizes all rotamers of each residue at every position.

To roughly illustrate the numbers of rotamers, in one version of Dunbrack & Karplus backbone-dependent, rotamer library, alanine has 1 rotamer, glycine has 1 rotamer, arginine has 55 rotamers, threonine has 9 rotamers, lysine has 57 rotamers, glutamic acid has 69 rotamers, asparagine has 54 rotamers, aspartic acid has 27 rotamers, tryptophan has 54 rotamers, glutamine has 69 rotamers, histidine has 54 rotamers, valine has 9 rotamers, isoleucine has 45 rotamers, leucine has 36 rotamers, methionine has 21 rotamers, serine has 9 rotamers, and phenylalnine has 36 rotamers.

As well be appreciated by those in the art, other rotamer libraries with all dihedral angles staggered can be used or generated.

In a preferred embodiment, at a minimum, at least one variable position has rotamers from at least two chemically different sidechains; that is, a sequence is being optimized, rather than a structure.

Once the group of potential rotamers is assigned for each variable residue position, the single-residue and pair-residue interactions are calculated for each residue. The single-residue and pair-residue interactions are combined to estimate the total rotamer interactions. This is shown as step 6 in FIG. 1. This step entails analyzing interactions of the rotamers to generate optimized polymer sequences. Simplistically, as is generally outlined above, the step comprises the use of a number of scoring functions, described below, to calculate energies of interactions of the rotamers, either to the backbone itself, to generic sidechains, or other rotamers. Hence, the scoring function calculate or estimate the energy contribution from various interactions which depend upon the conformation of a polymer.

Scoring Functions

The scoring functions utilized in this invention, a van der Waals potential scoring function, a hydrogen bond potential scoring function, a secondary structure propensity scoring function and an electrostatics scoring function, are readily apparent to those skilled in the art (Gasteiger, et al., Tetrahedron 1980, 36:3219-3288; Rappe, et al., J Phys. Chem. 1991, 95:3358-3363; Munoz et al., Current Op. in Biotech. 1995, 6:832; Minor, et al., Nature 1994, 367:660-663; Padmanabhan, et al., Nature 1990, 344:268-270; Munoz, et al., Protein Sci. 1994, 3:843). For example, the van der Waals scoring function is based on a van der Waals potential energy. There are a number of van der Waals potential energy calculations, including the well-known Lennard-Jones 12/6 potential with radii and well depth parameters. Electrostatic scoring functions may include coulombic interactions, dipole interactions and quadropole interactions.

In preferred embodiments, the energy of solvation is directly proportional to the exposed-nonpolar-surface area of a polymer. The present invention improves upon known methods for calculating the solvent-exposed area of polymers by incorporating the many-residue burial effect directly in the estimates of the single-residue and pair-residue areas as discussed in detail below.

In a preferred embodiment, the hydrogen bond potential consists of a distance-dependent term and an angle-dependent term (see Gasteiger, supra; see Rappe, supra).

In a preferred embodiment, a secondary structure propensity scoring function is used that is based on the specific sidechains, and is conformation independent (see Munoz, supra; see Minor, supra; see Padmanabhan, supra).

In a preferred embodiment, at least one scoring function is used for each variable residue position; in preferred embodiments, two, three or four scoring functions are used for each variable residue position.

Typically, the interactions with the backbone or sidechain also include terms for bond rotation, bond torsion and bond length. In preferred embodiments, the bond lengths and angles of the rotamers and polymer backbone are based on force-field potentials. Furthermore, force-field potentials may provide the parameters values for the scoring functions (i.e. well depth and radii parameters of the van der Waals scoring function and the distance dependent and angle dependent terms of the hydrogen bonding scoring function) used in the present invention. Force-fields that may be used in the present invention are well known in the art and include, but are not limited to, CHARMM (Brooks, et al., J. Comp. Chem. 1983, 4:187-217; MacKerell, et al., The Encyclopedia of Computational Chemistry, 1998, 1:271-277 (Chirchester: John Wiley & Sons); Cornell, et al., J. Amer. Chem. Soc. 1995, 117:5179; Woods, et al., J Phys. Chem. 1995, 99:3832-3846; Weiner, et al., J. Comp. Chem. 1986, 7:230; and Weiner, et al., J. Amer. Chem. Soc. 1984, 106:765; and Mayo, et al., J Phys. Chem. 1990, 94:8897).

5.2 Solvation Energy

In a preferred embodiment, the energy of solvation is directly proportional to the solvent-exposed nonpolar area of the polymer. As discussed above, the present invention improves upon known methods for calculating the solvent-exposed area of proteins by incorporating the many-residue burial effect directly in the estimations of the single-residue and pair-residue areas. In a preferred embodiment, the single-residue and pair-residue areas are estimated by taking into account the presence of not only the backbone but also generic sidechains.

Historically, the difficulty in obtaining an accurate pairwise approximation to surface areas lies in treating multiple-atom, multiple-residue buried areas. For two spheres in contact, there is a simple analytical formula to calculate the buried area (and therefore the exposed area) using the two radii and the separation between the spheres (Wodak, et al., J Proc. Natl. Acad. Sci. 1980, 77:1736-1740). For proteins, this formula was initially applied to each pair of atoms, and the total exposed and buried areas were estimated via a statistical combination (see Wodak et al., supra). Street and Mayo significantly improved on this statistical combination of pair-atom areas by calculating pair-residue areas, which has allowed use of faster search algorithms such as dead-end elimination (DEE) for design optimization.

Street and Mayo significantly improved on prior known methods for the statistical combination of pair-atom areas by calculating pair-residue areas (Street, et al., Fold. Des. 1998, :253-258). In the Street and Mayo method, two residues were placedat positions i and j, first separately and, then together, and the areas buried by the two sidechains were estimated by taking into account the presence of the backbone. Because buried areas often overlap, a straightforward addition of pair-residue areas overestimates the total buried area. Consequently, Street and Mayo scaled down the pairwise buried area by an adjustable factor. They went further and used three pairwise scaling factors for the residues classified into core and non-core: 0.42 for the interaction of core/core residues, 0.79 for non-core/non-core residues, and 0.74 for core/non-core residues. Street and Mayo classified residues as core or non-core using an algorithm that considered the direction of each sidechains's C_{α}-C_{β} vector relative to a surface computed using only the template C_{α} atoms with a carbon radius of 1.95 Å, a probe radius of 8 Å and no add-on radius. A residue was classified as a core position if both the distance from its C_{α} atom (along its C_{α}-C_{β} vector) to the surface was greater than 5.0 Å and the distance from its C_{β} atom to the nearest point in the surface was greater than 2.0 Å (Marshall, et al., J. Mol. Biol. 2001, 305:619-631). The scaling factor for the two core residues was smaller than the scaling factors with non-core residues involved because the overlapping burial effect is stronger in the core (see Street et al, supra). Despite the use of specialized scaling factors, the accuracy of the Street and Mayo method was still limited by the effect of overlapping burial of core residues.

The present invention improves upon known methods for calculating the solvent-exposed area of polymers by incorporating the many-residue burial effect directly in the estimations of the single-residue and pair-residue areas. Instead of calculating these areas by taking into account the presence of the backbone alone, the present invention calculates the areas by taking into account the presence of the backbone and generic sidechains. Each of these generic sidechains may consist of one or a number of spheres and may be optimized to approximate closely the presence of real sidechains. The generic sidechains may be optimized for a subset of polymers whose buried or exposed areas are known, or for a subset of polymers whose buried or exposed areas can be easily calculated utilizing exact methods described below. The optimized generic sidechains may then be utilized to predict the buried or exposed areas of polymers whose exposed or buried areas are unknown. The essential advantage of the present invention is that much of the overlapping burial effect is automatically accounted for by the genericized sidechains. As a result, errors in the area calculations are dramatically reduced, and there is no need to optimize scaling factors—they can be set to one.

The present invention is also directed to providing estimates of the solvent-exposed surface areas of nucleic acids. In this case “backbone” should be read as sugar-phosphate backbone and “side-chains” as nucleotide bases.

Street and Mayo Method.

Street and Mayo (Street, et al., Fold. Des. 1998; 3:253-258) used the following two pairwise formulas to calculate the total buried and exposed surface areas, respectively, of a protein (see FIG. 3(A-C) for a schematic representation):

$$\begin{matrix}{A}_{pairwise}^{buried} = {\sum }_{i}^{} ( {A}_{i , GXG}^{exposed} - {A}_{i , bb}^{exposed} ) + s {\sum }_{i < j}^{} ( {A}_{i , bb}^{exposed} + {A}_{j , bb}^{exposed} - {A}_{ij , bb}^{exposed} ) , & ( 1 ) \\ {A}_{pairwise}^{exposed} = {\sum }_{i}^{} {A}_{i , bb}^{exposed} - s {\sum }_{i < j}^{} ( {A}_{i , bb}^{exposed} + {A}_{j , bb}^{exposed} - {A}_{ij , bb}^{exposed} ) & ( 2 )\end{matrix}$$

Here

$${A}_{i , GXG}^{exposed}$$

is the exposed surface area of the i-th residue in the presence of the local tripeptide termed “GXG”. G-G stands for the backbone units of the previous and the following residues and X stands for the i-th residue. For the local tripeptide, Street and Mayo used [Cα, C, O]ᵢ₋ₗ, . . . ,[N,Cα]ᵢ₊₁,

$${A}_{i , bb}^{exposed}$$

is the exposed surface area of the i-th residue estimated taking into account the presence of the entire protein backbone, and

$${A}_{ij , bb}^{exposed}$$

is the combined exposed surface area of the i-th and j-th residues in the presence of the backbone. Then,

$${A}_{i , GXG}^{exposed} - {A}_{i , bb}^{exposed}$$

is the area of the i-th residue buried by parts of the backbone other than the local tripeptide structure. It is common in calculating protein surface areas to exclude the area buried by the local tripeptide because the portion of the surface buried due to local covalent bonds does not change during folding (Kurochkin, et al., Protein Eng. 1995, 8:437-442.

$${A}_{i , bb}^{exposed} + {A}_{j , bb}^{exposed} - {A}_{ij , bb}^{exposed}$$

is then the area buried by the proximity of the two residues at i and j, excluding that buried by the backbone (see FIG. 3C). The factor s, positive and less than one, scales down the pair-residue buried area to account for the effect of overlapping burial, i.e., that the same portion of a residue surface is buried by more than one sidechain or by the backbone and at least one sidechain.

In FIG. 3D, we show schematically the overlap of three sidechains at i, j, and k using the Street and Mayo method. The multiply-buried area (dotted line) would be exaggerated by Eqs. 1 and 2 if a unit scaling factor s=1.0 were used.

In addition, in Eqs. 1 and 2, the contributions from the polar (p) atoms, nitrogen and oxygen, and the nonpolar (n) atoms, carbon and sulfur can be separately estimated. The result is six total areas:

$${A}_{pairwise}^{buried} , {A}_{pairwise}^{exposed} , {A}_{pairwise}^{( p ) buried} , {A}_{pairwise}^{( p ) exposed} , {A}_{pairwise}^{( n ) buried} , {A}_{pairwise}^{( n ) exposed} ,$$

which can be compared with the six corresponding exact values (i.e., the values estimated using the “exact” method).
Generic-Sidechain Method.

In contrast to previous methods, the present invention estimates single-residue and pair-residue exposed surface areas taking into account the presence of generic sidechains in addition to the backbone.

$${A}_{i , gs}^{exposed}$$

is used to denote the exposed surface area of the i-th “residue” in the presence of the backbone but also the presence of other sidechains (in a genericized fashion to avoid overexpansion of variables to be considered) at all positions other than i. FIG. 4A shows a schematic representation of

$${A}_{i , gs}^{exposed}$$

depicted as a rectangle 30 against the backbone 40 and at residue position i of the backbone. All other sidechains are genericized and illustrated as circles 50 in FIG. 4A. To obtain a pairwise formula for the exposed surface area of residue i, we define

$${A}_{i : j , gs}^{exposed}$$

as the exposed surface area of the i-th residue taking into account the presence of (i) a nongenericized (“real”) sidechain at j, (ii) the backbone, and (iii) generic sidechains at all positions other than i and j (see FIG. 4B-C). Then,

$${A}_{i : j , gs}^{exposed} - {A}_{i , gs}^{exposed}$$

is the correction to the exposed area of the amino acid residue at i. This correction is necessary because of the influence in the calculation of

$${A}_{i : j , gs}^{exposed}$$

of the real sidechain at j (see FIG. 4B) in place of a generic one (see FIG. 4A). When the j-th residue is large and occupies a larger area than a generic sidechain at j would have been assumed to occupy, the correction

$${A}_{i : j , gs}^{exposed} - {A}_{i , gs}^{exposed}$$

is negative, to reduce the initial overestimate provided by

$${A}_{i , gs}^{exposed}$$

of the exposed area of residue i (see FIG. 4B) since it is now partially covered by the bulky sidechain j. On the other hand, when the j-th residue is small and occupies a smaller area than a generic sidechain at j,

$${A}_{i : j , gs}^{exposed} - {A}_{i , gs}^{exposed}$$

is positive, to increase the initial underestimate provided

$${A}_{i , gs}^{exposed}$$

by of the exposed area of residue i (see FIG. 4C). We therefore obtain the following exposed area formula for the i-th residue:

$${A}_{i , pairwise}^{exposed} = {A}_{i , gs}^{exposed} + s {\sum }_{j \neq i}^{} ( {A}_{i : j , gs}^{exposed} - {A}_{i , gs}^{exposed} ) \qquad ( 3 )$$

where, the first term is the single-residue approximation using generic sidechains at all other positions, and the second term sums up all corrections each using one additional real sidechain at a second position j and generic sidechains at all positions other than i and j. Of course, the position j varies each time, so a different previously generic sidechain is taken into account as a “real” side chain.

The scaling factor s accounts for any residual effects of overlapping burial; in practice, however, we can use s=1.0 as optimal, i.e., there is no systematic overestimate or underestimate of exposed areas. In other words, the present invention incorporates the many-residue burial effect directly in the single-residue and pair-residue areas. The total exposed area of the protein

$${A}_{pairwise}^{exposed}$$

is obtained by summing Eq. 3 over all residues i.

Similarly, it is straightforward to derive a “buried”, i.e. not exposed, area formula for the i-th residue

$${A}_{i , pairwise}^{buried} = ( {A}_{i , GXG}^{exposed} - {A}_{i , gs}^{exposed} ) + s {\sum }_{i , j \neq i}^{} ( {A}_{i , gs}^{exposed} - {A}_{i : j , gs}^{exposed} ) \qquad ( 4 )$$

where

$${A}_{i , GXG}^{exposed} - {A}_{i , gs}^{exposed}$$

is the area buried by the backbone and the generic sidechains, but not by the local tripeptide GXG, and

$${A}_{i , gs}^{exposed} - {A}_{i : j , gs}^{exposed}$$

is the correction to the area buried by the j-th residue. This correction is necessary because of the influence in

$${A}_{i , gs}^{exposed}$$

of the real sidechain at j (see FIG. 4B) in place of a generic one (FIG. 4A). Note that it is common in calculating protein surface areas to exclude the area buried by the local tripeptide because the burial due to local covalent bonds does not change during folding (Kurochkina, et al., Protein Eng. 1995, 8:437-442). The scaling factor s accounts for the residual effects of overlapping burial for the total buried area; in practice, however, s=1.0 is optimal, i.e., there is no systematic overestimation or underestimation of exposed areas. In other words, the present invention incorporates the many-residue burial effect directly in the single-residue and pair-residue areas.

It is convenient to manipulate Eq. 4 into similar form as Eq. 3. We define

$${A}_{i}^{total}$$

as the simple sum of the surface areas of all atoms in residue i without regard to burial or exposure, and

$${A}_{i , GXG}^{buried}$$

as the area of the i-th residue buried by the local tripeptide (GXG).
From this definition, we have,

$${A}_{i , GXG}^{exposed} = {A}_{i}^{total} - {A}_{i , GXG}^{buried} \qquad ( 5 )$$

and we define the following additionally buried areas excluding those buried by GXG,

$$\begin{matrix}{A}_{i , gs}^{buried} = {A}_{i}^{total} - {A}_{i , GXG}^{buried} - {A}_{i , gs}^{exposed} , & ( 6 ) \\ {A}_{i : j , gs}^{buried} = {A}_{i}^{total} - {A}_{i , GXG}^{buried} - {A}_{i : j , gs}^{exposed} , & ( 7 )\end{matrix}$$

Using Eqs. 5, 6, and 7, the expression for buried area is Eq. 4 can be written as

$${A}_{i , pairwise}^{buried} = {A}_{i , gs}^{buried} + s {\sum }_{j \neq i}^{} ( {A}_{i : j , gs}^{buried} - {A}_{i , gs}^{buried} ) \qquad ( 8 )$$

which is of the same form as the exposed area formula of Eq. 3. The total buried area of the protein

$${A}_{pairwise}^{buried}$$

is obtained by summing Eq. 8 over all residues i (i=1−n). Eqs. 3 and 8 are used extensively in our calculations.

In alternative embodiments, single-residue and pair-residue surface areas of a polymer are estimated by taking into account the presence of both the polymer backbone and at least one generic sidechain. For example, the exposed surface area of the i-th residue is estimated taking into account the presence of the backbone but also taking into account the presence of at least one other generic sidechain at a position(s) other than i. The exposed surface of the i-th residue is estimated by taking into account both the presence of (i) a nongenericized (“real”) sidechain at j, (ii) the backbone, and (iii) at least one generic sidechains at positions other than i and j.

In another alternative embodiment, the single-atom surface areas estimated taking into account both the backbone and generic sidechains for at least one atom. For example, the single-atom surface areas may be estimated for polar atoms only. Alternatively, the single-atom surface areas may be estimated for nonpolar atoms only. In preferred embodiments, the single-atom surface areas are estimated for both nonpolar and polar atoms.

In alternative embodiments, the single-residue and pair-residue surface areas of a polymer complex are estimated by taking into account the presence of both the polymer backbone and at least one generic sidechain. For example, the solvent-exposed surface area of a ligand bound to a protein may be determined.

In a preferred embodiment, the Richards definition of solvent-accessible surface area (Lee, et al., J. Mol. Biol. 1971, 55:379-400, hereby incorporated by reference) is used, with a probe radius ranging from 0.8 to 1.6 Å., with 1.4 Å being preferred, and Drieding van der Waals radii, scaled from 0.8 to 1.0. The accessible surface of a protein, according to Lee and Richard, is mapped out by the probe sphere center as it rolls over the protein. Carbon and sulfur are considered nonpolar. Nitrogen and oxygen are considered polar. Surface areas are estimated with the Connolly algorithm using a dot density of 10 Å⁻² (Connolly, (1983) (supra), hereby incorporated by reference). In the Connolly dot-surface algorithm, a set of approximately evenly spaced dots is placed on the extended van der Waals surface of each and every atom of a given protein, where the extended van der Waals radius is the sum of the atomic van der Waals radius and the probe sphere radius. If a dot on the extended van der Waals surface of a given atom lies outside the extended van der Waals surface of all other atoms, the dot is designated as a surface dot. The number of such surface dots for the surface area of atoms can be summed to give the accessible surface area of a residue, domain, protein, or protein assembly.

In an alternative embodiment, three scaling parameters are used corresponding to core/core, non-core/non-core, and core/non-core interactions as used in the core/non-core protein classification scheme of Marshall and Mayo (Marshall, et al., J. Mol. Biol. 2001, 305:619-631). For example, at each residue position on the backbone, three spheres with radius 2.0 Å plus the water add-on radius of 1.4 Å are placed at 1.53 Å, 2×1.53 Å, and 3×1.53 Å from the C, atom in the C_{α}-C_{β} direction. The exposed surface area of each three-sphere sidechain is then calculated taking into account the presence of the backbone and all other three-sphere sidechains. If the surface area of the three-sphere sidechain at a particular site is less than 24 Å² then this position is classified as a core position, else a non-core position.

In another embodiment, three-residue corrections to the buried areas are provided by calculating the exposed surface of residue i in the presence of two real sidechains at j and j′, and generic sidechains at all other positions. The three-body terms, however, would add significantly to the number of calculations required, and would not permit fast graph-based optimization techniques such as DEE (Desmet, et al., Nature 1992, 356:539-542; Moret, B. M. E., Algorithms from P to NP (Benjamin, Calif., 1991); Nemhauser, et al., Optimization 1989, New York, N.Y.; Bolon, et al., Proc. Natl. Acad. Sci. 2001, 98:14272-14279). So three-body calculations should be reserved for circumstances in which the extra accuracy justifies the extra effort, such as re-ranking of best pairwise solutions, or estimation of pairwise error.

In preferred embodiments, the generic sidechains are chosen from a group of simple geometric shapes and mixtures thereof. Most preferably as shown in FIG. 6, the generic sidechains are modeled as spheres described by three parameters: n, the number of spheres; d, the distance of the first sphere from a backbone atom and also the separation between the additional spheres (when it is assumed that the same residue will have a generic sidechain represented by more than one sphere, with each additional sphere “stacked” on the previous one); r, the radius of each sphere. The generic sidechain is then the n spheres of radius r placed at d, 2d . . . nd.

5.3 Side Chain Sampling and Optimization

Accordingly, as outlined above, the single-residue energy is the sum of the energies of each scoring function used at a particular position and the pair-residue energy is the sum of each scoring function used to evaluate every possible pair of rotamers. The scoring functions utilized comprise a van der Waals potential scoring function, a hydrogen bond potential scoring function, an atomic solvation scoring function, a secondary structure propensity scoring function and an electrostatics scoring function. Once calculated each single-residue energy for each possible rotamer is stored and each pair-residue energy for each possible rotamer pair is stored such that they may be used in subsequence calculations to determine the optimized polymer sequence. This is shown as step 8 in FIG. 1. As will be appreciated by those in the art, a global optimized sequence, is one that has the lowest energy of any possible sequence. Search algorithms that can be used to determine the optimized polymer sequence are well known in the art and include stochastic algorithms, such as Monte Carlo (See, Hellinga, et al., Proc. Natl. Acd. Sci. 1994, 91:5803-5807); Jiang, et al., Protein Sci. 2000, 9:403-416; Cootes, et al., J. Chem. Phys. 2000, 113:2489-2496; and genetic algorithms (see Jones, Prot. Sci. 1994, 3:567-574; Desjarlais, et al., Protein Sci. 1995, 4:2006-2018), and deterministic methods, such as the Dead End Elimination (see, Dahiyat, et al. Protein Sci. 1996, 5:895-903; Desmet, et al., Nature 356:539-542) and Self Consistent Mean Field Approach (see, Kono, et al., Proteins: Struct. Funct. Genet. 1994, 19:244-255; Bowie, et al., Science 1991, 253:164-170).

In a preferred embodiment, the designed proteins are chemically synthesized as is known in the art. Once made, the designed proteins are experimentally evaluated and tested for structure, function and stability, as required. In a preferred embodiment, the experimental results are used for design feedback and design optimization.

5.4 Implementation Systems and Methods

Computer System.

The analytical methods described in the previous subsections may preferably be implemented by the use of one or more computer systems, such as those described herein. Accordingly, FIG. 5 schematically illustrates an exemplary computer system suitable for implementation of the analytical methods of this invention. Computer 60 is illustrated here as comprising internal components linked to external components. However, a skilled artisan will readily appreciate that one or more of the components described herein as “internal” may, in alternative embodiments, be external. Likewise, one or more of the “external” components described here may also be internal. The internal components of computer system in this example include processor element 70 interconnected with a main memory 80. For example, in one preferred embodiment computer system 60 may be a Silicon Graphics R10000 Processor running at 195 MHz or greater and with 2 gigabytes or more of physical memory. In another, less preferable, exemplary embodiment, computer system 60 may be an Intel Pentium based processor of 150 MHz or greater clock rate and the 32 megabytes or more of main memory.

The external components may include a mass storage 90. This mass storage may be one or more hard disks which are typically packaged together with the processor and memory. Such hard disks are typically of at least 1 gigabyte storage capacity, and more preferably have at least 5 gigabytes or at least 10 gigabytes of storage capacity. The mass storage may also comprise, for example, a removable medium such as, a CD-ROM drive, a DVD drive, a floppy disk drive (including a Zip™ drive), or a DAT drive or other. Other external components include a user interface device 100, which can be, for example, a monitor and a keyboard. In preferred embodiments the user interface is also coupled with a pointing device 110 which may be, for example, a “mouse” or other graphical input device (not illustrated). Typically, computer system 60 is also linked to a network link 120, which can be part of an Ethernet or other link to one or more other, local computer systems (e.g., as part of a local area network or LAN), or the network link may be a link to a wide area communication network (WAN) such as the Internet. This network link allows computer system 60 to communicate with one or more other computer systems.

Typically, one or more software components are loaded into main memory 80 during operation of computer system 60. These software components may include both components that are standard in the art and components specifically adapted to the present invention, and the components collectively cause the computer system to function according to the analytical methods of the invention. Typically, the software components are stored on mass storage 90 (e.g., on a hard drive or on removable storage media such as on one or more CD-ROMs, RW-CDs, DVDs, floppy disks or DATs). Software component 130 represents an operating system, which is responsible for managing computer system 60 and its network interconnections. The operating system may be a conventional one, and may be, for example, a UNIX operating system or, less preferably, a member of the Microsoft Windows™ family of operating systems (for example, Windows 2000, Windows Me, Windows 98, Windows 95 or Windows NT) or a Macintosh operating system. Software component 140 represents common languages and functions conveniently present in the system to assist programs implementing the methods specific to the invention. Languages that may be used include, for example, C, C++ and less preferably JAVA. The analytical methods of the invention may also be programmed in mathematical software packages which allow symbolic entry of equations and high-level specification of processing, including algorithms to be used, thereby freeing a user of the need to procedurally program individual equations and algorithms. Examples of such packages include Matlab from Mathworks (Natick, Mass.), Mathematica from Wolfram Research (Champaign, Ill.) and S-Plus from Math Soft (Seattle, Wash.). Accordingly, software component 150 represents the analytic methods of the invention as programmed in a procedural language or symbolic package. The memory 80 may, optionally, further comprise software components 160 which cause the processor to calculate or determine a three-dimensional structure for a macromolecule and, in particular, for a given polymer sequence such as a protein or nucleic acid sequence. Such programs are well known in the art, and numerous software packages are publicly available including, for example, Rosetta. The memory may also comprise one or more other software components, such as one or more other files representing, e.g., one or more sequences of polymer residues including, for example, a parent sequence and/or other sequences (for example, mutant sequences). The memory 80 may also comprise one or more files representing the three-dimensional structures of one or more sequences, including a file representing the three-dimensional structure of a parent sequence, such as a parent protein or nucleic acid. Computer Program Products.

The present invention also provides computer program products which can be used, e.g., to program or configure a computer system for implementation of analytical methods of the present invention. A computer program product of the invention comprises a computer readable medium such as one or more compact disks (i.e., one or more “CDs”, which may be CD-ROMs or a RW-CDs), one or more DVDs, one or more floppy disks (including, for example, one or more ZIP™ disks) or one or more DATs to name a few. The computer readable medium has encoded thereon, in computer readable form, one or more of the software components 150 that, when loaded into memory 80 of a computer system 60, cause the computer system to implement analytic methods of the present invention. The computer readable medium may also have other software components encoded thereon in computer readable form. Such other software components may include, for example, functional languages 140 or an operating system 130. The other software components may also include one or more files or databases including, for example, files or databases representing one or more polymer sequences (e.g. protein or nucleic acid sequences) and/or files or databases representing one or more three-dimensional structures for particular polymer sequences (e.g., three-dimensional structures for proteins and nucleic acids.

System Implementation.

In an exemplary implementation, to practice the methods of the present invention the polymer backbone sequence with at least one variable residue position may first be loaded into the computer system 60. For example, the polymer backbone sequence may be directly entered by a user from monitor and keyboard 100 and by directly typing a sequence of code of symbols representing different backbone atoms or a specific name for the polymer backbone sequence. Alternatively, a user may specify the polymer backbone sequence, e.g., by selecting a sequence from a menu of candidate backbone sequences presented on the monitor or by entering an accession number for a sequence in a database (for example, the GenBank or SWISPROT database) and the computer system may access the selected polymer backbone sequence from the database, e.g., by accessing a database in memory 80 or by accessing the sequence from a database over the network connection, e.g., over the internet.

A rotamer library may then be loaded into the computer system 60. For example, the rotamer library may be directly entered by a user from monitor and keyboard 100. Alternatively, a user may specify the rotamer library, e.g., by selecting a rotamer library from a menu of candidate rotamer libraries presented on the monitor and the computer system may access the selected rotamer library from the database, e.g., by accessing a database in memory 80 or by accessing the sequence from a database over the network connection, e.g., over the internet.

The programs then cause the computer system to calculate the single-residue and pair-residue energies for each rotamer at each position. The matrix of single-residue and pair-residue energies is stored. For example, the memory 80 or mass storage 90 of the computer may store the results of the single-residue and pair-residue energies. Finally the programs may cause the computer system to obtain an optimized three-dimensional structure of the polymer. For example, the software components may, themselves, calculate a three-dimensional structure using the optimization search algorithms (i.e. Monte Carlo, Dead-End Elimination or Self Consistent mean Field Approach).

Alternative systems and methods for implementing the analytic methods of this invention are also intended to be comprehended within the accompanying claims. In particular, the accompanying claims are intended to include the alternative program structures for implementing the methods of this invention that will be readily apparent to those skilled in the relevant art(s).

### 6. EXAMPLES

The present invention is also described by means of particular examples. However, the use of such examples anywhere in the specification is illustrative only and in no way limits the scope and meaning of the invention or of any exemplified term. Likewise, the invention is not limited to any particular preferred embodiments described herein. Indeed, many modifications and variations of the invention will be apparent to those skilled in the art upon reading this specification and can be made without departing from its spirit and scope. The invention is therefore to be limited only by the terms of the appended claims along with the full scope of equivalents to which the claims are entitled.

6.1 Pairwise Surface-Area Calculations

In particular, the Example set forth in section 6.1 describes a preferred embodiment of a procedure for estimating single-residue and pair-residue surface areas in a polypeptide. This description is not limited to any particular protein or sequence of amino acid residues. The procedure is described in general terms for a polypeptide of N residues, where each residue involved in the calculation is denoted by a subscript, e.g. i or j. This algorithm therefore can be readily applied to any sequence of amino acid residues of interest to the user. Moreover, the procedure can be applied to other polymers and molecular assemblies beside polypeptides. These include, but are not limited to, nucleic aicds, carbohydrates, and branched polymers, such as poly-4-methyl pentene-1.

This example also describes a preferred embodiment of a procedure for calculating the exact surface area for a residue and a preferred embodiment for specifying the generic sidechains in a polypeptide. Again, these descriptions are not limited to any particular protein or sequence of amino acid residues. The described procedures for the exact surface area calculations and the specification of generic sidechains are utilized in estimating single-residue and pair-residue surface areas in a polypeptide.

Exact Surface-Area Calculations

To calculate the exact surface areas for the i-th residue, we evaluate the buried area of each atom m⁽ⁱ⁾ in this residue, including backbone atoms (N, C_{α}, C, and O) and sidechain atoms. Hydrogen atoms are not considered in any of our surface calculations. The van der Waals radii of the atoms are 1.5 Å for nitrogen, 1.4 Å for oxygen, 1.85 Å for sulphur, 1.5 Å for carbonyl carbons, and 2.0 Å for other carbons. For each atom, 256 evenly distributed dots are imagined on the spherical surface (Shrake, et al., J. Mol. Biol. 1973, 79:351-371) to serve as testing points, as to whether they lie on a buried or an exposed part of the surface. The radius of the sphere is the van der Waals radius plus a water “add-on” radius of 1.4 Å representing the radius of a water molecule. As described above, the inclusion of the water add-on radius enables the calculation of the solvent accessible surface area, excluding those regions too small for a water molecule to penentrate as part of the solvent-exposed surface area of the protein. For each atom m⁽ⁱ⁾ we find which of the 256 dots are buried by any other atom p of the protein (where the water radius is also added to the radius of each atom p). If p is one of the atoms in the tripeptide GXG, i.e., p is one of the N, C_{α}, C, O backbone atoms of the previous or the next residue or one of the other atoms in residue i, then the dots on the surface of m⁽ⁱ⁾ covered by p are marked as “buried” by GXG; if not, they are marked as “regularly” buried (i.e. buried by backbone, not the GXG, or sidechains). As noted above, it is common in calculating protein surface areas to exclude the area buried by the local tripeptide because the burial due to local covalent bonds does not change during folding (see Kurochikin et al., supra).

The marking of buried points is done using the mask method of (disclosed in LeGrand, et al., J. Comp. Chem. 1992, 14:349-352). In Legrand and Merz's mask algorithm, a set of points is placed evenly on the surface of a unit sphere centered at the origin (for example, 256, 512, 1024, or 2048 points). Each point is represented by a bit in a multi-byte word. A set of unit probe spheres is then placed at positions around the origin at a distance from zero to two, representing all spheres intersecting with the sphere centered at the origin. A multi-byte word (mask) records points on the original sphere covered by a particular probe sphere (the corresponding bits in the mask are set to zero, while the reminaing bits are one). For each atom in a multi-atom molecule, a binary AND operation of masks corresponding to the other atoms is a quick operation which yields the exposed points on the atom's surface. The essence of the mask algorithm is therefore to store spherical intersection properties in masks, and to use the fast binary AND operation to mark buried and exposed points on the surface of one sphere.

Thus, after considering all other atoms of each amino acid residue of the protein, the 256 dots on the surface of atom m⁽ⁱ⁾ are divided into three categories: unmarked, marked as buried by local tripeptide GXG, and marked as regularly buried. Counting the dots in each of these three categories then gives respectively the three areas, the atom exposed area

$${a}_{{m}^{( i )} , exact}^{exposed} ,$$

the atom area buried by local tripeptide GXG,

$${a}_{{m}^{( i )} , GXG}^{buried}$$

and the atom “regulary” buried area

$${a}_{{m}^{( i )} , exact}^{buried} .$$

By summing these three quantities over all atoms m⁽ⁱ⁾ in residue i we obtain the residue exposed area

$${A}_{{m}^{( i )} , exact}^{exposed} ,$$

the residue area buried by local tripeptide GXG

$${A}_{{m}^{( i )} , GXG}^{buried} ,$$

and the residue “regularly” buried area

$${A}_{{m}^{( i )} , exact}^{exposed}$$

If we sum over only the polar atoms in the residue (nitrogen and oxygen), we obtain the polar atoms exposed area

$${A}_{{m}^{( i )} , exact}^{( p ) exposed} ,$$

the polar atoms area buried by local tripeptide GXG, Aₘ_{⁽ⁱ⁾}_{,GXG}^{(p)buried} and the polar atoms “regularly” buried area

$${A}_{{m}^{( i )} , exact}^{( p ) buried} .$$

If we sum over only the nonpolar atoms (carbon and sulphur) we obtain the nonpolar atoms exposed area

$${A}_{{m}^{( i )} , exact}^{( n ) exposed} ,$$

the nonpolar atoms area buried by the local tripeptide GXG

$${A}_{{m}^{( i )} , GXG}^{( n ) buried} ,$$

and the nonpolar atoms “regularly” buried area

$${A}_{{m}^{( i )} , exact}^{( n ) buried} .$$

Specification of Generic Sidechains.

To use our pairwise method to calculate surface areas, we first need to specify generic sidechains. We consider these generic sidechains to be described by three parameters: n, the number of spheres per generic sidechain; d, the distance of the first sphere from C_{α} in the direction from C_{α} to C_{β} of the ith residue and also the separation between the spheres in the same generic sidechain; r, the radius of each sphere. Given the atomic coordinates of a protein backbone, virtual C_{β} atoms are placed at standard glycine-C_{β} positions. The generic sidechain is then the number of n spheres of radius r placed at d, 2d, . . . ,nd from C_{α} in the direction from C_{α} to C_{β} of the ith residue.

Pairwise Surface-Area Calculations

We calculate the single-residue and pair-residue surface area metrics needed for the pairwise method as follows. As in the exact surface-area calculation, for each atom m⁽ⁱ⁾ in residue i, we determine which of the 256 dots are buried by all other atoms in residue i, the entire backbone, and the generic sidechains at positions other than i. Note that the water add-on radius of 1.4 Å is also added to the generic-sidechain radius r. We obtain three atomic areas

$${a}_{{m}^{( i )} , gs}^{exposed} , {a}_{{m}^{( i )} , GXG}^{buried} ,$$

and

$${a}_{{m}^{( i )} , gs}^{buried} ,$$

which are defined above, and, when summed over all atoms in residue i give the single-residue quantities for the ith residue:

$${A}_{i , gs}^{exposed} , {A}_{i , GXG}^{buried} , and {A}_{i , gs}^{buried} .$$

Note that in this model the area buried by the local tripeptide,

$${A}_{i , GXG}^{buried} ,$$

is an exact quantity and does not depend on the generic sidechains. If we sum the other two areas over only the polar atoms in the residue, we obtain

$${A}_{i , gs}^{( p ) exposed}$$

and

$${A}_{i , gs}^{( p ) buried} ;$$

and if we sum over only the nonpolar atoms, we obtain

$${A}_{i , gs}^{( n ) exposed}$$

and

$${A}_{i , gs}^{( n ) buried} .$$

Next, we consider real residues at both i and j. For each atom m⁽ⁱ⁾ in i, we individually consider all other atoms in residue i, all atoms in residue j, the entire backbone, and the generic sidechains at positions other than i and j. We thus obtain a

$${a}_{{m}^{( i )} : j , gs}^{exposed}$$

and

$${a}_{{m}^{( i )} : j , gs}^{buried} ,$$

which when summed over all atoms in residue i give the pair-residue quantities:

$${A}_{i : j , gs}^{exposed} and {A}_{i : j , gs}^{buried} .$$

If we sum over only the polar atoms in the residue, we obtain

$${A}_{i : j , gs}^{( p ) exposed}$$

and

$${A}_{i : j , gs}^{( p ) buried} ;$$

and if we sum over only the nonpolar atoms, we obtain

$${A}_{i : j , gs}^{( n ) exposed}$$

and

$${A}_{i : j , gs}^{( n ) buried} .$$

These single-residue and pair-residue quantities can then be combined in Eqns. 3 and 8 to give

$${A}_{i , pairwise}^{exposed} , {A}_{i , pairwise}^{buried} , {A}_{i , pairwise}^{( p ) exposed} , {A}_{i , pairwise}^{( p ) buried} , {A}_{i , pairwise}^{( n ) exposed} , and {A}_{i , pairwise}^{( n ) buried} .$$

When i is summed over all residues, we obtain the six total areas:

$${A}_{pairwise}^{exposed} , {A}_{pairwise}^{buried} , {A}_{pairwise}^{( p ) exposed} , {A}_{pairwise}^{( p ) buried} , {A}_{pairwise}^{( n ) exposed} ,$$

and

$${A}_{pairwise}^{( n ) buried} .$$

If the total number of residues in a given protein is N, then for each residue i there are N−1 metrics to calculate, corresponding to each j ≠i. But in fact, most of the A_{i:j,gs} are identical to A_{i,gs} because the residue j is not in contact with residue i. In practice, we first calculate the N single-residue quantities A_{i,gs}, as described above, and then for each residue j, we check all atoms in i to see whether any of them is in contact with either an atom in j or the generic sidechain at j. If not, we know A_{i:j,gs}=A_{i,gs}. This simple check saves a great deal of computation time, as, for proteins of several hundred residues, we find that often less than five percent of the N(N−1) residue pairs are in fact in contact and influence the exposed area or conversely the buried area. The calculations are performed on a cluster of 32 nodes (1.26 GHz Pentium processors) running Sycld Beowulf cluster operating system.

6.2 Measures of Error

The Example set forth in section 6.2 describes a preferred embodiment of a procedure for testing the accuracy of the procedures described in the Example presented in Section 6.1, supra. In particular, the procedures described in the Example presented in Section 6.1, supra, for estimating the solvent-exposed surface area of a polypeptide are compared to the exact solvent-exposed surface area of the polypeptide. This description is not limited to any particular protein or sequence of amino acid residues. The procedure is described in general terms for a polypeptide of N residues, where each residue involved in the calculation is denoted by a subscript, e.g. i or j. This algorithm therefore can be readily applied to any sequence of amino acid residues of interest to the user. Moreover, the procedure can be applied to other polymers and molecular assemblies beside polypeptides. These include, but are not limited to, nucleic aicds, carbohydrates, and branched polymers, such as poly-4-methyl pentene-1.

To compare the exact and pairwise surface-area results for each protein (or protein domain) of length N, we use the following measure of error:

$$δ {A}_{protein} ( k ) = \frac{1}{N} {\sum }_{i = 1}^{N} ( {A}_{i , pairwise} ( k ) - {A}_{i , exact} ( k ) ) \qquad ( 9 )$$

which is the average deviation per residue for the protein k. To compare exact and pairwise results on a residue-by-residue basis, we use the following measure of error:
δA_{residue}(i)=A_{i,pairwise}−A_{i,exact} (10)
which is the deviation for a single residue i.

The first formula measures the error for the entire surface of a protein. The accuracy of pairwise results for total area are reflected in the average and standard deviation of δAₚᵣₒₜₑᵢₙ over sets of proteins. The second formula measures the error in the surface area of individual residues. The accuracy of pairwise results for residue-by-residue areas are reflected in the average and standard deviation of δA_{residue} over sets of residues. These two measures can be applied to the six different areas: total buried, polar buried, nonpolar buried, total exposed, polar exposed, and nonpolar exposed.

6.3 Test Set and Optimization of Generic-Sidechain Parameters

This example demonstrates the use of the present invention to calculate surface area for actual protein sequences. In particular, the algorithm described in Section 6.1, supra, is used here to calculate the solvent-exposed surface area for ten different proteins: (1) 1enh, (2) 1pga, (3) 1ubi, (4) 1mol, (5) 1kpt, (6) 4azu-A, (7) 1gpr, (8) 1gcs, (9) 1edt, and (10) 1pbn. The results obtained for the ten protein set are compared to the results reported by Street and Mayo (see Street et al., supra) for the same ten protein set. The data presented here demonstrates that the algorithm and procedure of this invention are able to better estimate the solvent-exposed surface area of a polypeptide while, without adding a substantial computational burden.

To further demonstrate the efficiency of the algorithm described in Section 6.1, supra, the algorithm is used here to calculate the solvent-exposed surface area for a 377 single-protein representative domains from the CATH database. The results obtained for the 377 single-protein set demonstrate that the algorithm and procedure of this invention can be utilized for larger more comprehensive set of proteins.

This example further tests the accuracy of the predictions for the computational experiments described in Section 6.1, supra. In this example the predictions for the computational experiments described in 6.1, supra, are compared to the predictions for the method of Street and Mayo (see Street et al., supra) for a 1 0-protein set and a CATH protein set. This example also describes the optimization of generic-sidechain parameters described in the Example presented in Section 6.1, supra. Optimization of Generic-Sidechain Parameters As a test, we first studied the same 10-protein set used by Street and Mayo: (1) 1enh, (2) 1pga, (3) 1ubi, (4) 1mol, (5) 1kpt, (6) 4azu-A, (7) 1gpr, (8) 1gcs, (9) 1edt, and (10) 1pbn (see Street et al., supra). In order to compare to Street and Mayo, we present results for total buried area and nonpolar exposed area. Our generic-sidechain parameters n, d, r, and the scaling factor s were determined by minimizing

$$δ {A}^{( 1 )} = \sqrt{\frac{1}{10} {\sum }_{k = 1}^{10} {[ δ {A}_{protein}^{buried} ( k ) ]}^{2}} \qquad ( 11 )$$

which is the root-mean-square value of the average total protein deviation per residue δAₚᵣₒₜₑᵢₙ for buried area for the 10-protein set. We chose to minimize

$$δ {A}_{protein}^{buried}$$

rather than

$$δ {A}_{residue}^{buried}$$

(the residue-by-residue deviation) to make sure that the total buried areas are as accurate as possible. Note also that by minimizing δA⁽¹⁰⁾, we minimized both the average and the spread of

$$δ {A}_{protein}^{buried}$$

for the 10-protein set. For n=0, i.e., the Street and Mayo method without generic sidechains, we minimized δA⁽¹⁰⁾ with respect to one parameter, the scaling factor s. For n=0, we also implemented the version of Street and Mayo's method with three different scaling factors: for i and j both core residues, for i and j both non-core residues, and for i or j a core residue and the other a non-core residue.

To compare with the Street and Mayo's three-parameter method, we have used the core/non-core classification scheme of Marshall and Mayo (see Marshall et al., supra). Namely, at each residue position on the backbone, three spheres with radius 2.0 Å plus the water add-on radius 1.4 Å, are placed at 1.53 Å, 2×1.53 Å, and 3×1.53 Å from the C_{α} atom in the Cα-C_{β} direction. The exposed surface area of each three-sphere sidechain is then calculated taking into account the presence of the backbone and all other three-sphere sidechains. If the surface area of the three-sphere sidechain at a particular site is less than 24 Å² then this position is classified as a core position, else a non-core position.

For n=1, 2, and 3 generic-sidechain spheres, we fixed s=1.0 and minimized δA⁽¹⁰⁾ over the spacing d and the radius r of the spheres. We were able to use a scaling, factor s=1.0 because the generic sidechains already account for all systematic effects of overlapping burial. For n=0, using one parameter s, the best result is

$$δ {A}_{best}^{( 10 )} = 2.22 {Å}^{2}$$

for S=0.67. For n=0, using three parameters, the best result is

$$δ {A}_{best}^{( 10 )} = 0.70 {Å}^{2}$$

for s=0.55 (core/core), 0.77 (non-core/non-core), and 0.73 (core/non-core) (the difference from Street and Mayo parameters, 0.42, 0.79, and 0.74, respectively, is due to our use of a slightly modified definition of surface and core residues). Street and Mayo utilized [C., C, O]ᵢ₋₁, . . . ,[N,C_{α}]ᵢ₊1 for the local tripeptide whereas we have used [N, C_{α}, C, O]ᵢ₋₁,[N,C_{α}, C, O]ᵢ₊₁. For our generic-sidechain method with a fixed s=1.0, we obtained:

$$\begin{matrix}δ {A}_{best}^{( 10 )} = 0.43 {Å}^{2} for n = 1 , d = 2.10 {Å}^{2} , r = 3.09 Å ; \\ = 0.47 {Å}^{2} for n = 2 , d = 1.09 {Å}^{2} , r = 2.92 Å ; \\ = 0.48 {Å}^{2} for n = 3 , d = 0.61 {Å}^{2} , r = 2.85 {Å}^{2} .\end{matrix} \qquad ( 10 )$$

These are the generic-sidechain parameters used in the following calculations. In FIG. 6, we show the three generic sidechains drawn to scale with optimized parameters d. and r.

The computational time required for the present invention scales in the same way with protein length as for the method of Street and Mayo. However, the use of a spherical generic sidechain in the present invention increases the computational time by about 20 percent.

Results for the 10-Protein Set and CATH proteins

FIG. 7 shows surface-area results for the 10-protein set of Street and Mayo. For each protein, panel (a) shows the difference between the total buried area using our pairwise method,

$${A}_{pairwise}^{( n ) buried}$$

and the exact and the exact value,

$${A}_{exact}^{( n ) buried}$$

versus the exact value, and panel (b) shows the difference between the nonpolar exposed area using the pairwise method

$${A}_{pairwise}^{( n ) exposed} ,$$

and the exact value,

$${A}_{exact}^{( n ) exposed} ,$$

versus the exact value. Total-area results without generic sidechains are shown both for a single scaling factor s=0.67, and for Street and Mayo's three-parameter scaling. For total areas, our new method, with one, two, or three-sphere generic sidechains, and no scaling parameter, achieves significantly better results. FIGS. 7C and 7D show the distributions of the residue-by-residue area deviation δA_{residue} (Eq 10) for the 10 proteins, respectively for total buried and nonpolar exposed area. It is clear that our new method also achieves significantly and consistently better results for residue-by-residue surface areas than the methods without generic sidechains. The average and standard deviation results of both δAₚᵣₒₜₑᵢₙ and δA_{residue} for the 10-protein set are given in Table I below.

We have further tested our generic-sidechain method on a larger, more systematic set of proteins: 377 single-segment representative domains from the CATH database. The CATH database is a hierarchical domain classification of protein structures in the Brookhaven protein databank. All non-protein, model, and “C-alpha only” structures are not included in CATH. Only crystal structures solved to resolution better than 3.0 Å are considered, together with NMR structures. There are four major levels in this hierarchy; Class, Architecture, Topology (fold family) and Homologous superfamily. We use the 377 CATH classification (Release 2.4) T-level (topology) single-segment representative domains. In this level there are altogether 775 representative domains, of which 663 are single-segment domains; of these 663, we skip (1) 16 for which the PDB files are not found in the Protein Data Bank, (2) 6 in which ACE (acetyl group) or PCA (pyroglutamic acid) are included in the domain, (3) 112 for which some atomic coordinates are not found in the PDB files, and (4) 152 for which the domain definition is not consistent (Jones, et al., Structure 1997, 5:1093-1108). The results are presented in FIG. 8 and Table I, as shown below. The single-segment domains that are skipped are therefore either those that do not have all coordinate information need in surface calculation or those whose domain definition is not consistent (for example, the domain in the protein 1 LEA is specified to have 84 residues but the starting position of the domain is give as 1 and the stopping position 72). Clearly, as compared to the optimized three-parameter method without generic sidechains, our new, generic-sidechain method achieves significantly better results. In particular, the average total protein deviation per residue for buried area

$$δ {A}_{protein}^{buried}$$

has been reduced in magnitude from −1.43 Å² to 0.38 Å², and the spread of this average deviation from protein to protein has been reduced from 1.96 Å² to 0.58 Å². Strikingly, the spread of the residue-by-residue buried-area deviation

$$δ {A}_{residues}^{buried}$$

has been reduced from 7.42 Å² to 3.70 Å². Thus, for individual residues, the use of generic sidechains reduces errors by more than a factor of two. Similar error reductions are also achieved for exposed areas. The calculations described above are performed on a cluster of 32 nodes (1.26 GHz Pentium processors) running Sycld Beowulf cluster operating system.

TABLE I: Average and standard deviation of δAₚᵣₒₜₑᵢₙ and δA_{residue} in Å² for the 10-protein set and the CATH protein set, for the method without generic sidechains and one scaling parameter (rows 1; n=0, s=0.67), without generic sidechains and three scaling parameters (rows 2; n=0, s=0.55, 0.77, 0.73), and the method with generic sidechains, with one (rows 3; n=1, d=2.10 Å, r=3.09 Å, s=1.0), two (rows 4; n=2, d=1.09 Å, r=2.92 Å, s=1.0), or three (rows 5; n=3, d=0.61 Å, r=2.85 Å, s=1.0) spheres.

Average and standard deviation of δAₚᵣₒₜₑᵢₙ and δA_{residue} in Å² for the
10-protein set and the CATH protein set, for the method without generic sidechains and
one scaling parameter (rows 1; n = 0, s = 0.67), without generic sidechains and three
scaling parameters (rows 2; n = 0, s = 0.55, 0.77, 0.73), and the method with generic
sidechains, with one (rows 3; n = 1, d = 2.10Å, r = 3.09Å, s = 1.0), two (rows 4;
n = 2, d = 1.09Å, r = 2.92Å, s =1.0), or three (rows 5; n = 3, d = 0.61Å, r = 285Å,
s = 1.0) spheres.

| | ${δA}_{protein}^{buried}$ | ${δA}_{protein}^{( n ) exposed}$ | ${δA}_{protein}^{residue}$ | ${δA}_{residue}^{( n ) exposed}$ |
|---|---|---|---|---|
| 10-protein set | | | | |
| n = 0, s =0 0.67 | 0.10 ± 2.21 | −0.196 ± 1.67 | 1.11 ± 13.2 | −2.68 ± 11.4 |
| n = 0, s = 0.55, 0.77, 0.73 | 0.05 ± 0.70 | −0.91 ± 0.68 | 0.13 ± 7.01 | −0.90 ± 5.94 |
| n = 1, d = 2.10 Å, r = 3.09 Å, s = 1.0 | −0.01 ± 0.43 | 0.04 ± 0.24 | −0.04 ± 3.58 | 0.11 ± 2.65 |
| n = 2, d = 1.09 Å, r = 2.92 Å, s = 1.0 | −0.08 ± 0.47 | 0.07 ± 0.25 | −0.13 ± 3.47 | 0.15 ± 2.58 |
| n = 3, d = 0.61 Å, r = 2.85 Å, s = 1.0 | 0.06 ± 0.48 | −0.07 ± 0.26 | 0.06 ± 3.58 | −0.06 ± 2.81 |
| CATH set | | | | |
| n = 0, s = 0.67 | −2.84 ± 3.21 | 0.97 ± 2.72 | −1.79 ± 11.1 | 0.15 ± 9.57 |
| n = 0, s = 0.55, 0.77, 0.73 | −1.43 ± 1.96 | 0.51 ± 1.63 | −1.06 ± 7.42 | 0.25 ± 6.31 |
| n = 1, d = 2.10 Å, r = 3.09 Å, s = 1.0 | 0.38 ± 0.58 | −0.18 ± 0.42 | 0.31 ± 3.70 | −0.12 ± 2.88 |
| n = 2, d = 1.09 Å, r = 2.92 Å, s = 1.0 | 0.35 ± 0.59 | −0.16 ± 0.43 | 0.28 ± 3.63 | −0.11 ± 2.85 |
| n = 3, d = 0.61 Å, r = 2.85 Å, s = 1.0 | 0.41 ± 0.62 | −0.23 ± 0.45 | 0.38 ± 3.75 | −0.20 ± 3.02 |

6.4 Test of Efficiency

This example demonstrates that the increased efficiency of the present invention is intimately related to the present invention's ability to incorporate the many-residue burial effect directly in the estimations of the single-residue and pair-residue areas. The present invention incorporates the many-residue burial effect by taking into account the presence of generic sidechains during the estimations of the single-residue and pair-residue areas.

In FIG. 9A, we show the average and in FIG. 9C the standard deviation of the residue-by-residue buried area deviation for each type of amino acid, averaged over the 377 CATH proteins. The generic-sidechain method significantly improves the accuracy of the area calculation for all 20 amino-acid types. For the two methods without generic sidechains, the largest errors are associated with the largest hydrophobic amino acids: phenylalanine (F), isoleucine (I), leucine (L), methionine (M), tryptophan (W), tyrosine (Y), and, for the s=0.67 case, with the medium-sized valine (V). These large hydrophobic amino acids are typically found in the protein core, where the overlapping burial effect is most severe. It is precisely for these amino acids that the generic-sidechain method achieves the greatest fractional reduction in error. This clearly demonstrates that the success of the generic-sidechain method is due to its improved treatment of overlapping buried areas. The calculations described above are performed on a cluster of 32 nodes (1.26 GHz Pentium processors) running Sycld Beowulf cluster operating system.

6.5 Test of Sensitivity of Optimization Area

This example verifies the predictions made in the Example presented in Section 6.3, supra, are independent of the choice of area optimized.

The success of the generic-sidechain method does not depend sensitively on the choice of area optimized. As described in the Example presented in Section 6.3, we optimized parameters using the total buried area for the 10-protein set of Street and Mayo. For comparison, in FIGS. 9B and 9D, we show results for parameters chosen to optimize total nonpolar exposed area for the same protein set. The generic-sidechain parameters are very similar, and, more importantly, the residue-by-residue results for buried area are equally good. In contrast, the results without generic sidechains (n=0) depend significantly on the choice of area optimized: in FIG. 10B, when optimizing the nonpolar exposed area, the nonpolar residues (for example, F, I, L) have small errors but the polar residues (for example, E, K, R) have large errors; in FIG. 10A, when optimizing total buried area, the polar and nonpolar residues on average have similar errors (but with opposite signs).

As one can see in FIGS. 7, 8, and 9, the one-sphere generic sidechains do as well as those with two or three spheres. In FIG. 6, with the three optimized sidechains drawn to scale, we can see that these three generic sidechains are geometrically similar and thus lead to similar results.

1. A method of estimating polymer surface areas pairwise in terms of constituent residues comprising:
(A) calculating single-residue and pair-residue areas for each residue position taking into account the presence of both the polymer backbone and of a generic sidechain at one or more other residue positions;
(B) determining exposed and buried areas by a combination of these single-residue and pair-residue areas.

2. A method of claim 1, wherein the polymer sequence comprises a sequence of amino acid residues.

3. A method of claim 1, wherein the polymer sequence comprises a sequence of nucleotide residues.

4. A method for estimating polymer surface areas as in claim 1, further comprising estimating surface areas for one or more individual residues.

5. A method for estimating polymer surface areas as in claim 1, wherein at least one generic sidechain is different from at least one other generic sidechain, said sidechains being employed at different residue positions.

6. A method for estimating polymer surface areas as in claim 1, wherein generic sidechains intended to model particular amino acids, including particular conformations of these amino acids, are employed at at least one residue position.

7. A method for estimating polymer surface areas as in claim 1, wherein the generic sidechains are chosen from among a general class of simple geometric shapes, and positioned in relation to the polymer backbone, in such a manner as to minimize the deviation of the estimated surface area of the polymer from the exact surface area of the polymer.

8. A method for estimating polymer surface areas as in claim 1, wherein at least one of the generic sidechains comprises one or more spheres.

9. A method of estimating a polymer assembly surface area pairwise in terms of constituent residues comprising:
(A) calculating single-residue and pair-residue areas for each residue position taking into account the presence of both the polymer backbones and of a generic sidechain at one or more other residue positions;
(B) determining exposed and buried areas by a combination of these single-residue and pair-residue areas.

10. A method of claim 9, wherein the polymer sequence comprises a sequence of amino acid residues.

11. A method of claim 9, wherein the polymer sequence comprises a sequence of nucleotide residues.

12. A method for estimating polymer surface areas as in claim 9, further comprising estimating surface areas for one or more individual residues.

13. A method for estimating polymer surface areas as in claim 9, wherein at least one generic sidechain is different from at least one other generic sidechain, said sidechains being employed at different residue positions.

14. A method for estimating polymer surface areas as in claim 9, wherein generic sidechains intended to model particular amino acids, including particular conformations of these amino acids, are employed at at least one residue position.

15. A method for estimating polymer surface areas as in claim 9, wherein the generic sidechains are chosen from among a general class of simple geometric shapes, and positioned in relation to the polymer backbone, in such a manner as to minimize the deviation of the estimated surface area of the polymer from the exact surface area of the polymer.

16. A method for estimating polymer surface areas as in claim 9, wherein at least one of the generic sidechains comprises one or more spheres.

17. A computer system for estimating the buried or exposed surface area of a polymer or polymer assembly, which computer system comprises: memory and a processor interconnected with the memory and having one or more software components loaded therein, wherein the one or more software components cause the processor to execute steps of method according claim 1.

18. A computer program comprising a computer readable medium having one or more software components encoded in computer readable form, wherein the one or more software components may be loaded into a memory of a computer system and cause a processor interconnected with the memory to execute steps of a method according to claim 1.

19. A method of estimating polymer surface areas pairwise, the polymer comprising residues wherein each residue comprises a backbone unit and a real sidechain, each of the backbone units being linked by a covalent bond to the next adjacent backbone unit, said linked backbone units forming a polymer backbone, said backbone unit and real sidechains comprising nonpolar and polar atoms, the method comprising:
(A) calculating and summing the single-atom areas for each atom in residue i taking into account the presence of both the polymer backbone and generic sidechains at one or more residue positions other than i to obtain single-residue areas;
(B) calculating and summing the single-atom areas for each atom in residue i taking into account the presence of the polymer backbone, all the atoms in residue j, and generic sidechains at one or more residue positions other than i and j to obtain pair-residue areas;
(C) determining exposed and buried areas by a combination of these single-residue and pair-residue areas.

20. A method of claim 19, wherein the polymer comprises a sequence of amino acid residues.

21. A method of claim 19, wherein the polymer comprises a sequence of nucleotide residues.

22. A method for estimating polymer or polymer assembly surface areas as in claim 19, wherein the single-atom areas of residue i from steps (A-B) are summed for polar atoms only.

23. A method for estimating polymer surface areas as in claim 19, wherein the single-atom areas of residue i from steps (A-B) are summed for nonpolar atoms only.

24. A method for estimating polymer or polymer assembly surface areas as in claim 19, wherein at least one generic sidechain is different from at least one other generic sidechain, said sidechains being employed at different residue positions

25. A method for estimating polymer or polymer assembly surface areas as in claim 19, wherein generic sidechains intended to model particular amino acids, including particular conformations of these amino acids, are employed at at least one residue position.

26. A method for estimating polymer or polymer assembly surface areas as in claim 19, wherein the generic sidechains are chosen from among a general class of simple geometric shapes, and positioned in relation to the polymer backbone, in such a manner as to minimize the deviation of the estimated surface area from the exact surface area.

27. A method for estimating polymer or polymer assembly surface areas as in claim 19, wherein at least one of the generic sidechains comprises one or more spheres.

28. A computer system for estimating the buried or exposed surface area of a polymer, which computer system comprises:
memory and a processor interconnected with the memory and having one or more software components loaded therein, wherein the one or more software components cause the processor to execute steps of method according claim 19.

29. A computer program comprising a computer readable medium having one or more software components encoded in computer readable form, wherein the one or more software components may be loaded into a memory of a computer system and cause a processor interconnected with the memory to execute steps of a method according to claim 19.

30. A method for estimating an exposed surface area for a residue i of a polymer having N residues,
wherein each of said residues comprises a backbone unit and a real sidechain, with the backbone units of each residue being linked by a covalent bond to the backbone unit of the next available residue such that the linked backbone units form a polymer backbone;
which method comprises steps of:
(A) calculating a single-residue exposed surface area for residue i, accounting for the presence of (a) the real sidechain of i-residue, (b) the backbone, and (c) genericized sidechains at all residues other than the residue i;
(B) calculating, for each residue identified by the index j, a pair-residue exposed surface area of the i-residue, accounting for the presence of (a) the real sidechains of the i and j residues, (b) the backbone, and (c) genericized sidechains at all residues other than the residues i and j;
(C) subtracting, for each residue j, the single residue exposed surface area for the i-residue from the pair-residue exposed surface area of the i-residue, to obtain a surface-area correction for the i-residue due to residue j;
(D) summing, for all residues j, the surface-area correction for the i-residue due to residue j to obtain a total corrected surface area for the residue i; and
(E) adding the total corrected surface area for the residue i to single-residue exposed surface area for the residue i to obtain an estimate of the exposed surface area for the residue i.

31. A method of claim 30, farther comprising the step of summing, for each residue i, the exposed surface area for the residue i to obtain the total exposed surface area of the polymer.

32. A method of claim 31, wherein the polymer comprises a sequence of amino acid residues.

33. A method of claim 31, wherein the polymer comprises a sequence of nucleotide residues.

34. A method for estimating polymer surface areas as in claim 30, wherein generic sidechains intended to model particular amino acids, including particular conformations of these amino acids, are employed at at least one residue position.

35. A method for estimating polymer surface areas as in claim 30, wherein the generic sidechains are chosen from among a general class of simple geometric shapes, and positioned in relation to the polymer backbone, in such a manner as to minimize the deviation of the estimated surface area of the polymer from the exact surface area of the polymer.

36. A method for estimating polymer surface areas as in claim 30, wherein at least one of the generic sidechains comprises one or more spheres.

37. A method of claim 30, wherein a fraction of the total corrected surface area for residue i is added to the single-residue exposed surface for the residue i to obtain the exposed surface for the residue i.

38. A method for estimating the exposed surface area for a residue i of a polymer having N residues,
wherein each residue comprises a variable M atoms wherein each atom comprises part of a backbone unit or part of the sidechain, each of the backbone units being linked by a covalent bond to the next adjacent backbone units, said linked backbone units forming a polymer backbone;
which method comprises steps of:
(A) calculating a first single-atom exposed surface area of the kth atom of the i-residue accounting for the presence of (a) the backbone, and (b) genericized sidechains at all residue other than i;
(B) summing the first single-atom exposed surface area for all atoms in residue i to obtain a single-residue exposed surface area for residue i;
(C) calculating a second single-atom exposed surface area of the kth atom of the i-residue taking into account the presence of (a) a real sidechain of j-residue, (b) the backbone, and (c) genericized sidechains for all residues other than i and j, wherein 1<j<N;
(D) summing the second single-atom exposed surface area for all atoms in residue i to obtain a pair-residue exposed surface area for residue i;
(E) subtracting, for each residue j, the single residue exposed surface area for the i-residue from the pair-residue exposed surface area of the i-residue, to obtain a surface area correction for the i-residue due to residue j;
(F) summing, for all residues j, the surface-area correction for the i-residue due to residue j to obtain a total corrected surface area for the residue i; and
(G) adding the total corrected surface area for the residue i to single-residue exposed surface area for the residue i to obtain an estimate of the exposed surface area for the residue i.

39. A method of claim 38 further comprising the step of summing, for each residue i, the exposed surface area for the residue i to obtain the exposed surface area of the polymer.

40. A method of claim 39, wherein the polymer comprises a sequence of amino acid residues.

41. A method of claim 39, wherein the polymer comprises a sequence of nucleotide residues.
